(19) United States
(12) Patent Application Publication
(10) Pub. No.: US 2024/0424245 A1
(43) Pub. Date: Dec. 26, 2024

(54) SYSTEMS AND METHODS FOR PERFORMING BEHAVIOR DETECTION AND BEHAVIORAL INTERVENTION

(71) Applicant: QUALCOMM Incorporated, San Diego, CA (US)

(72) Inventors: Daniel James GUEST, San Diego, CA (US); Wen CHEN, Shanghai (CN); Katheryn Victoria RAMP, Oakland, CA (US); Yujie DAI, Shanghai (CN)

(21) Appl. No.: 18/682,867

(22) PCT Filed: Oct. 29, 2021
(86) PCT No.: PCT/CN2021/127351

§ 371 Date: Feb. 9, 2024

(51) Int. Cl. A61M 21/00; G16H 20/70

(52) U.S. Cl. CPC A61M 21/00; G16H 20/70; A61M 2021/005; A61M 2209/088

(57) ABSTRACT

Systems and techniques are described for predicting one or more behaviors and generating one or more interventions based on the behavior(s). For instance, a system (e.g., an extended reality (XR) device) can obtain behavioral information associated with a user of the XR device and can determine, based on the behavioral information, a likelihood of the user engaging in a behavior. The system can determine, based on the determined likelihood exceeding a likelihood threshold, an intervention. The system can generate (e.g., display or otherwise output) the intervention. The system can determine, subsequent to generating the intervention, whether the user engaged in the behavior and can determine an effectiveness of the intervention based on whether the user engaged in the behavior. The system can send, to a server, an indication of the effectiveness of the intervention for use in determining interventions for one or more additional users.

### CROSS-REFERENCE TO RELATED APPLICATIONS

This application for Patent is a 371 of international Patent Application PCT/CN2021/127351, filed Oct. 29, 2021, which is hereby incorporated by referenced in its entirety and for all purposes.

### FIELD

The present disclosure generally relates to behavior detection and behavioral intervention. In some examples, aspects of the present disclosure are related to systems and techniques for performing behavior detection and intervention to influence behaviors.

### BACKGROUND

Chronic conditions are a leading cause of death and disability in the world, contributing to approximately two-thirds of the global burden of disease with enormous healthcare costs for societies and governments.

A majority of chronic disease can be attributed to lifestyle factors and therefore are preventable by forming healthy behaviors. For example, among U.S. adults, more than 90% of type-2 diabetes, 80% of cardiovascular disease, 70% of stroke, and 70% of colon cancer are potentially preventable by a combination of non-smoking, maintaining healthy weight, performing moderate physical activity, maintaining a healthy diet, and adhering to moderate alcohol consumption. Undergoing behavioral changes is complex and takes time, requiring a person to disrupt a habitual lifestyle while simultaneously fostering a new, possibly unfamiliar set of actions.

### BRIEF SUMMARY

In some examples, systems and techniques are described for performing behavior detection and behavioral interventions for influencing user behaviors in accordance with a user's goals (or target behaviors). According to at least one illustrative example, a method of generating one or more interventions is provided. The method includes: obtaining, by an extended reality (XR) device, behavioral information associated with a user of the XR device; determining, by the XR device based on the behavioral information, a likelihood of the user engaging in a behavior; determining, by the XR device based on the determined likelihood exceeding a likelihood threshold, an intervention; generating, by the XR device, the intervention; determining, subsequent to generating the intervention, whether the user engaged in the behavior; determining an effectiveness of the intervention based on whether the user engaged in the behavior; and sending, to a server, an indication of the effectiveness of the intervention for use in determining interventions for one or more additional users.

In another illustrative example, an apparatus (e.g., an extended reality (XR) device) is provided for generating one or more interventions. The apparatus includes at least one memory (e.g., configured to store data, such as sensor data, one or more images, etc.) and at least one processor (e.g., implemented in circuitry) coupled to the at least one memory. The at least one processor configured to: obtain behavioral information associated with a user of the apparatus; determine, based on the behavioral information, a likelihood of the user engaging in a behavior; determine, based on the determined likelihood exceeding a likelihood threshold, an intervention associated with the behavior; generate the intervention; determine, subsequent to outputting the intervention, whether the user engaged in the behavior; determine an effectiveness of the intervention based on whether the user engaged in the behavior; and send, to a server, an indication of the effectiveness of the intervention for use in determining interventions for one or more additional users.

In another example, a non-transitory computer-readable medium is provided that has stored thereon instructions that, when executed by one or more processors, cause the one or more processors to: obtain behavioral information associated with a user of the XR device; determine, based on the behavioral information, a likelihood of the user engaging in a behavior; determine, based on the determined likelihood exceeding a likelihood threshold, an intervention associated with the behavior; generate the intervention; determine, subsequent to outputting the intervention, whether the user engaged in the behavior; determine an effectiveness of the intervention based on whether the user engaged in the behavior; and send, to a server, an indication of the effectiveness of the intervention for use in determining interventions for one or more additional users.

In another example, an apparatus (e.g., an extended reality (XR) device) for processing one or more frames is provided. The apparatus includes: means for obtaining behavioral information associated with a user of the XR device; means for determining, based on the behavioral information, a likelihood of the user engaging in a behavior; means for determining, based on the determined likelihood exceeding a likelihood threshold, an intervention associated with the behavior; means for generating the intervention; means for determining, subsequent to outputting the intervention, whether the user engaged in the behavior; means for determining an effectiveness of the intervention based on whether the user engaged in the behavior; and means for sending, to a server, an indication of the effectiveness of the intervention for use in determining interventions for one or more additional users.

In some aspects, the method, apparatuses, and computer-readable medium described above can include: sending, to the server, contextual information associated with the intervention, wherein the contextual information associated with the intervention comprises at least one of a time of day, one or more actions by the user of the XR device (or apparatus) prior to the intervention, the behavioral information associated with the user of the XR device (or apparatus), a location of the user of the XR device, and a proximity of the user of the XR device (or apparatus) to one or more individuals.

In some aspects, the method, apparatuses, and computer-readable medium described above can include: sending, to the server, one or more characteristics associated with the user of the XR device (or apparatus), wherein the one or more characteristics associated with the user of the XR device (or apparatus) comprise at least one of gender, age, family status, target behavior, country, culture, locale, personality type, one or more health conditions, one or more dietary restrictions, and one or more physical capabilities.

In some aspects, to obtain the behavioral information associated with the user of the XR device, the method, apparatuses, and computer-readable medium described above can include: determining one or more behavioral triggers that are predictive of the behavior, wherein the one or more behavioral triggers include at least one of a stress level of the user, a heart rate of the user, an object within a field of view of the XR device (or apparatus), a location at which the user is located, a time at which the behavioral information is obtained, one or more people in proximity to the user, and an activity in which the user is engaged.

In some aspects, to obtain the behavioral information associated with the user of the XR device (or apparatus), the method, apparatuses, and computer-readable medium described above can include: determining one or more pre-behaviors indicative of a likelihood of the user engaging in the behavior.

In some aspects, to obtain the behavioral information associated with the user of the XR device (or apparatus), the method, apparatuses, and computer-readable medium described above can include: detecting, in one or more images obtained by the XR device (or apparatus), one or more behavioral artifacts associated with the behavior.

In some aspects, to generate the intervention, the method, apparatuses, and computer-readable medium described above can include: displaying virtual content on a display of the XR device (or apparatus), wherein a real-world environment is viewable through the display of the XR device (or apparatus) as the virtual content is displayed by the display.

According to another illustrative example, a method of generating one or more interventions is provided. The method includes: obtaining, by a server, first intervention information associated with a first user and a first intervention; updating, based on the first intervention information, one or more parameters of an intervention library, wherein the one or more parameters of the intervention library are based at least in part on second intervention information associated with a second user and a second intervention; and determining a third intervention for a third user based on the updated one or more parameters of the intervention library.

In another illustrative example, a system (e.g., including at least one server) is provided for generating one or more interventions. The system includes at least one memory (e.g., configured to store data, such as sensor data, one or more images, etc.) and at least one processor (e.g., implemented in circuitry) coupled to the at least one memory. The at least one processor configured to: obtain first intervention information associated with a first user and a first intervention; update, based on the first intervention information, one or more parameters of an intervention library, wherein the one or more parameters of the intervention library are based at least in part on second intervention information associated with a second user and a second intervention; and determine a third intervention for a third user based on the updated one or more parameters of the intervention library.

In another example, a non-transitory computer-readable medium is provided that has stored thereon instructions that, when executed by one or more processors, cause the one or more processors to: obtain first intervention information associated with a first user and a first intervention; update, based on the first intervention information, one or more parameters of an intervention library, wherein the one or more parameters of the intervention library are based at least in part on second intervention information associated with a second user and a second intervention; and determine a third intervention for a third user based on the updated one or more parameters of the intervention library.

In another example, an apparatus for processing one or more frames is provided. The apparatus includes: means for obtaining first intervention information associated with a first user and a first intervention; means for updating, based on the first intervention information, one or more parameters of an intervention library, wherein the one or more parameters of the intervention library are based at least in part on second intervention information associated with a second user and a second intervention; and means for determining a third intervention for a third user based on the updated one or more parameters of the intervention library.

In some aspects, the first intervention information associated with the first user comprises at least one of an intervention type, an indication of an effectiveness of the first intervention, an intervention context associated with the first intervention, and one or more characteristics associated with the first user.

In some aspects, to determine the third intervention for the third user based on the updated one or more parameters of the intervention library, the method, apparatuses, and computer-readable medium described above can include: obtaining, by the server, third intervention information associated with the third user; determining a correlation between the third intervention information associated with the third user and fourth intervention information associated with the intervention library; determining, based on the correlation between the third intervention information and the fourth intervention information exceeding a correlation threshold, the third intervention; and sending, to a device associated with the third user, the third intervention.

In some aspects, the first intervention information comprises contextual information associated with the first intervention.

In some aspects, the contextual information associated with the first intervention comprises at least one of a time of day, one or more actions by the first user prior to the first intervention, the first intervention information associated with the first user, a location the first user, and a proximity of the first user to one or more individuals.

In some aspects, the first intervention information comprises one or more characteristics associated with the first user.

In some aspects, the one or more characteristics associated with the first user comprise at least one of gender, age, family status, target behavior, country, culture, locale, and personality type.

In some aspects, the method, apparatuses, and computer-readable medium described above can include: obtaining, by the server, fifth behavioral information associated with a fifth user and a fifth behavior; updating, based on the fifth behavioral information, one or more parameters of a behavior library, wherein the one or more parameters of the behavior library are based at least in part on sixth behavioral information associated with a sixth user; and determining one or more behavior parameters for a seventh user based on the updated one or more parameters of the intervention library.

In some aspects, the one or more behavior parameters for the seventh user comprise at least one of behavioral triggers, pre-behaviors, and behavioral artifacts associated with the fifth behavior.

In some aspects, the one or more behavior parameters for the seventh user comprise one or more weightings associated with determining a likelihood that the seventh user will perform or not perform the fifth behavior.

In some aspects, the fifth behavioral information comprises one or more characteristics associated with the fifth user.

In some aspects, the fifth behavioral information comprises contextual information associated with the fifth behavior.

In some aspects, one or more of the apparatuses described above is, is part of, or includes a mobile device (e.g., a mobile telephone or so-called “smart phone” or other mobile device), a wearable device, an extended reality device (e.g., a virtual reality (VR) device, an augmented reality (AR) device, or a mixed reality (MR) device), a personal computer, a laptop computer, a server computer, a vehicle (e.g., a computing device of a vehicle), or other device. In some aspects, an apparatus includes a camera or multiple cameras for capturing one or more images. In some aspects, the apparatus includes a display for displaying one or more images, notifications, and/or other displayable data. In some aspects, the apparatus can include one or more sensors. In some cases, the one or more sensors can be used for determining a location and/or pose of the apparatus, a state of the apparatuses, and/or for other purposes. In some aspects, the apparatus can include one or more microphones (e.g., for capturing auditory input and/or other sound or audio). In some aspects, the apparatus can include one or more speakers (e.g., for providing auditory feedback or other audio output).

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The foregoing, together with other features and embodiments, will become more apparent upon referring to the following specification, claims, and accompanying drawings.

### BRIEF DESCRIPTION OF THE DRAWINGS

Illustrative embodiments of the present application are described in detail below with reference to the following figures:

FIG. 1A through FIG. 1G are images illustrating example interventions, in accordance with some examples of the present disclosure;

FIG. 2 is a simplified block diagram illustrating an example extended reality (XR) system, in accordance with some examples of the present disclosure;

FIG. 3 is a diagram illustrating an example of an XR system being worn by a user, in accordance with some examples of the present disclosure;

FIG. 4 is a block diagram illustrating an example learning system, in accordance with some examples of the present disclosure;

FIG. 5 is a flow diagram illustrating an example of a process for generating one or more interventions, in accordance with some examples;

FIG. 6 is a flow diagram illustrating another example of a process for generating one or more interventions, in accordance with some examples;

FIG. 7 is a flow diagram illustrating an example of a process for predicting one or more behaviors, in accordance with some examples;

FIG. 8 is a block diagram illustrating an example of a deep learning network, in accordance with some examples;

FIG. 9 is a block diagram illustrating an example of a convolutional neural network, in accordance with some examples;

FIG. 10 is a diagram illustrating an example of a computing system for implementing certain aspects described herein.

### DETAILED DESCRIPTION

Certain aspects and embodiments of this disclosure are provided below. Some of these aspects and embodiments may be applied independently and some of them may be applied in combination as would be apparent to those of skill in the art. In the following description, for the purposes of explanation, specific details are set forth in order to provide a thorough understanding of embodiments of the application. However, it will be apparent that various embodiments may be practiced without these specific details. The figures and description are not intended to be restrictive.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes May be made in the function and arrangement of elements without departing from the scope of the application as set forth in the appended claims.

As noted previously, chronic conditions are a leading cause of death and disability in the world. Many of such chronic conditions can be attributed to lifestyle factors and can be prevented or managed through the implementation of healthy behaviors. A large percentage of chronic conditions (e.g., type-2 diabetes, cardiovascular disease, stroke, etc.) can potentially be prevented or improved by leading a healthy lifestyle, such as by not smoking, maintaining healthy weight, performing moderate physical activity, maintaining a healthy diet, and adhering to moderate alcohol consumption.

However, undergoing behavioral changes is complex and takes time, requiring a person to disrupt a habitual lifestyle while simultaneously fostering a new, possibly unfamiliar set of actions. It is difficult for people to rely on willpower alone to make successful behavioral change. A person tends to act in pursuit of what that person wants or needs at any given moment. A person's intentions and beliefs about what is good or bad only influence the person's actions if they create sufficiently strong desires to engage in the target behavior at the relevant moment.

There are techniques that can be used to help influence people to have healthier behaviors. A wide range of behavioral change techniques have been identified in psychological research, such as environmental restructuring (e.g., not having unhealthy snacks in the house, putting hand sanitizers where people can easily see and access them, etc.), prompts/cues (e.g., place a sticker on the door to remind oneself to take workout clothes and shoes for fitness classes after work), etc.

Systems, apparatuses, processes (also referred to as methods), and computer-readable media (collectively referred to as “systems and techniques”) are described herein for identifying behaviors that are likely to occur and identifying and generating behavioral interventions to influence the likely behaviors. For instance, using a computing device and contextual awareness technology, behavioral interventions adapted to specific situations faced by users can be delivered in real time (Just In Time Adaptive Intervention, JITAI) and more effectively (e.g., by selecting interventions better tailored to the user and/or providing interventions that are more difficult to ignore) to help users address moments of vulnerability for unhealthy behaviors or take opportunities to perform healthy behaviors. In one illustrative example, a multi-user intervention system (e.g., a server) can learn the effectiveness of interventions across different users. In some cases, the multi-user intervention system can learn the effectiveness of interventions across multiple different users. In some cases, learning the effectiveness of interventions across multiple different users can include determining effectiveness of interventions for subsets of users sharing similar characteristics and/or experiencing similar contexts. Additionally or alternatively, the multi-user intervention system can learn to predict user behaviors based on behavior information and indications of behavior prediction effectiveness of different users. In some aspects, learning to predict user behaviors can include determining the effectiveness of predicting user behavior for subsets of users sharing similar characteristics and/or experiencing similar contexts. The technical effect of a multi-user intervention system learning the effectiveness of interventions and/or learning to predict user behaviors across users includes, but is not limited to, providing interventions that are more likely to be effective, constraining interventions that may be ineffective for a particular user, more accurately predicting user behaviors, or the like.

In some cases, the systems and techniques can be performed using an extended reality (XR) system or device. XR systems or devices can provide virtual content to a user and/or can combine real-world or physical environments and virtual environments (made up of virtual content) to provide users with XR experiences. The real-world environment can include real-world objects (also referred to as physical objects), such as people, vehicles, buildings, tables, chairs, and/or other real-world or physical objects. XR systems or devices can facilitate interaction with different types of XR environments (e.g., a user can use an XR system or device to interact with an XR environment). XR systems can include virtual reality (VR) systems facilitating interactions with VR environments, augmented reality (AR) systems facilitating interactions with AR environments, mixed reality (MR) systems facilitating interactions with MR environments, and/or other XR systems. Examples of XR systems or devices include head-mounted displays (HMDs), smart glasses (e.g., AR glasses), among others. In some cases, an XR system can track parts of the user (e.g., a hand and/or fingertips of a user) to allow the user to interact with items of virtual content.

In some cases, an XR system can include an optical “see-through” or “pass-through” display (e.g., see-through or pass-through AR HMD or AR glasses), allowing the XR system to display XR content (e.g., AR content) directly onto a real-world view without displaying video content. For example, a user may view physical objects through a display (e.g., glasses or lenses), and the AR system can display AR content onto the display to provide the user with an enhanced visual perception of one or more real-world objects. In one example, a display of an optical see-through AR system can include a lens or glass in front of each eye (or a single lens or glass over both eyes). The see-through display can allow the user to see a real-world or physical object directly, and can display (e.g., projected or otherwise displayed) an enhanced image of that object or additional AR content to augment the user's visual perception of the real world.

See-through or pass-through XR systems are intended to be worn while the user is engaged with the real world (as opposed to VR, in which the user is immersed in virtual content and the real world is fully occluded). Unlike smartphones, PCs, and other computing devices, head-mounted XR devices (e.g., smart glasses, HMDs, etc.) are worn on the face and directly mediate the user's visual and auditory sensory channels.

In some examples, an XR system (or server in communication with the XR system) can identify behavioral information for a user of the XR system. As used herein, behavioral information can include behavioral triggers, pre-behaviors, behavioral artifacts, any combination thereof, and/or other behavioral information. The XR system or another device (e.g., a mobile device) can identify behavioral triggers that increase the likelihood that a user of the XR system will engage in a particular behavior (e.g., stress level of the user, a heart rate of the user, a location at which the user is located, a time at which the behavioral information is obtained, one or more people in proximity to the user, an activity in which the user is engaged, or other behaviors—in some cases, these may not be obvious to the user). The XR system (or the server or other device) can additionally or alternatively identify pre-behaviors (e.g., walking to a cabinet containing alcohol) for a user of the XR system. In some cases, the pre-behaviors can indicate that the user has already decided to engage in the unhealthy behavior or to avoid engaging in the healthy behavior. The XR system (or the server) can additionally or alternatively identify pre-behaviors and/or triggers based on attention tracking (e.g., detecting eye movement to measure visual attention) and/or based on measuring biometrics of the user (e.g., to identify cravings). In some cases, the XR system (or the server) can correlate the attention tracking and/or biometrics to actual behavior to validate a predictive model. The XR system (or the server) can additionally or alternatively identify behavioral artifacts, which can include physical objects associated with the behavior (e.g., alcohol bottle, pack of cigarettes, running shoes, etc.). In some cases, a user can input an action plan for one or more target behavior changes (e.g. I will not smoke after meals, I will not eat sugary foods after 4 pm, I will run three times a week in the morning, etc.), and the XR system can obtain the behavioral information noted above (e.g., pre-behaviors, behavioral triggers, and/or behavioral artifacts) for each target behavior change (e.g., behavioral information for quitting smoking, behavioral information for exercising more often, etc.).

The term “target behavior” is used herein to describe a behavior that is selected for the user to change, either by stopping or reducing the behavior (e.g., quit drinking alcohol), or by starting or increasing the behavior (e.g., exercise three days a week). In some cases, the target behavior can be selected by the user, and in some cases the target behavior can be selected by a third party, such as a physician, an insurance company, or the like.

The XR system (or the server) can use any combination of the behavioral information to determine a likelihood (e.g., using a machine-learning (ML) based model for each person) of a person engaging in a target behavior (or not engaging in the target behavior). For example, a person who is walking towards an alcohol cabinet has an increasing likelihood to consume an alcoholic beverage as they get closer to the alcohol cabinet. The XR system (or the server) can generate, and in some cases perform (e.g., display, output as audio, provide haptic feedback, and/or provide other feedback), an appropriate intervention based on a likelihood of the user to engage in the target behavior (or not engage in the target behavior), based on effectiveness of interventions (e.g., interventions that have worked or not worked) for that user or other users in the past at different times, based on user input indicating one or more interventions a user prefers, and/or based on other factors. In some implementations, the XR system (or the server) can send the generated intervention to be performed by another device. In some aspects, the generated intervention can range from no intervention to completely obscuring an object and alerting a designated support person. As used herein, references to generating an intervention can include an XR system outputting and/or performing the intervention to the user as well as the XR system sending the intervention to be output and/or performed by another device. In some cases, the intervention can become more intense or otherwise emphasized as the user gets closer to the behavioral artifacts. The intervention can be positive (e.g., a positive intervention with the message “Yes! Grab your shoes and go for a jog”) or can be negative (e.g., a negative intervention with the message “Put down the cigarettes”). In some cases, biometrics of the user (e.g., heart rate, temperature, etc.) can be analyzed to determine whether the user is likely to engage in the behavior.

In some examples, the XR system can utilize a learning system (e.g., which can be ML based, such as using one or more neural networks) to determine the most effective interventions across a plurality of users. The learning system can apply the learned interventions to the population of users based on individual characteristics. In some cases, the learning system can be implemented as one or more servers that are in communication with the XR system.

In some examples, the learning system can maintain an intervention library. In some examples, the learning system can score interventions on effectiveness. The learning system (e.g., using an ML model, such as one or more neural networks) can associate user characteristics, goals, and/or contextual information with likelihood of success of a particular intervention.

Examples of user characteristics can include gender, biological sex, age, family status, target behavior, severity of a particular problem (e.g., smoking, over eating, etc.), country/culture, previously effective interventions, personality type (e.g., fear response, disgust response, etc.), one or more current and/or past health conditions of the user (e.g., high blood pressure, weight issues, etc.), family medical history, one or more dietary restrictions of the user (e.g., lactose intolerant, high fiber diet, low calorie diet, etc.), one or more physical capabilities and/or limitations of the user (e.g., weak or injured back, paralysis, etc.), job type (e.g., shift work, daily fixed schedule, number of jobs), education level, content history (e.g. whether the user has viewed articles or videos related to the intervention and/or goal), health knowledge, previously effective interventions for friends and family, recent health behaviors (e.g. exercise, sleep, nutrition in the past day), personal preferences of the user (e.g., preferred types of food, preferred types of exercise, preferred types of interventions) social support, motivation level, any combination thereof, and/or other characteristics. The learning system can monitor if a particular intervention was or was not successful. The learning system can update the multi-user intervention library based on whether a particular intervention was or was not successful. In some examples, the learning system can be implemented on the XR system. In some examples, the learning system can send updates to a multi-user intervention library, which can also be referred to as a Global Intervention Efficacy Analysis (GIEA) server. In some cases, the learning system can be used to learn predictive behaviors for specific user types (e.g., demographic based, severity of problem, etc.).

Contextual information can include, without limitation, a time of day, one or more actions by the user prior to the intervention, the behavioral information associated with the user and/or the intervention, a location and/or environment associated with the location of the user (and/or the XR device) before and/or during the intervention, a proximity of the user to one or more individuals, and the combinations of technologies/sensors in use by the user at the time of the intervention. For example, the environment associated with the location of the user can include whether or not there are stairs a person can take, is the user in a kitchen, is there space available for the user to exercise, weather at the user's location (e.g., hot, cold, raining, windy, etc.), presence of a behavioral artifact (e.g., cigarettes, running shoes), etc.

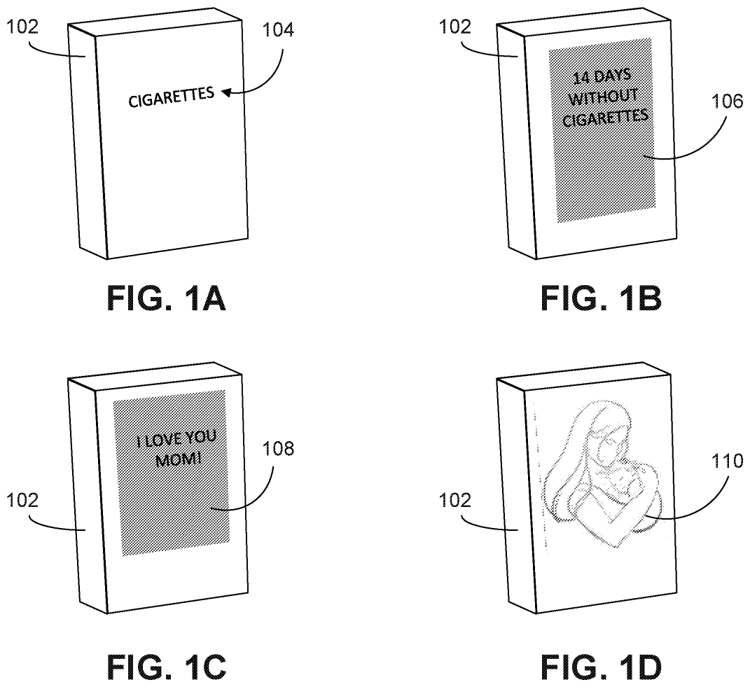

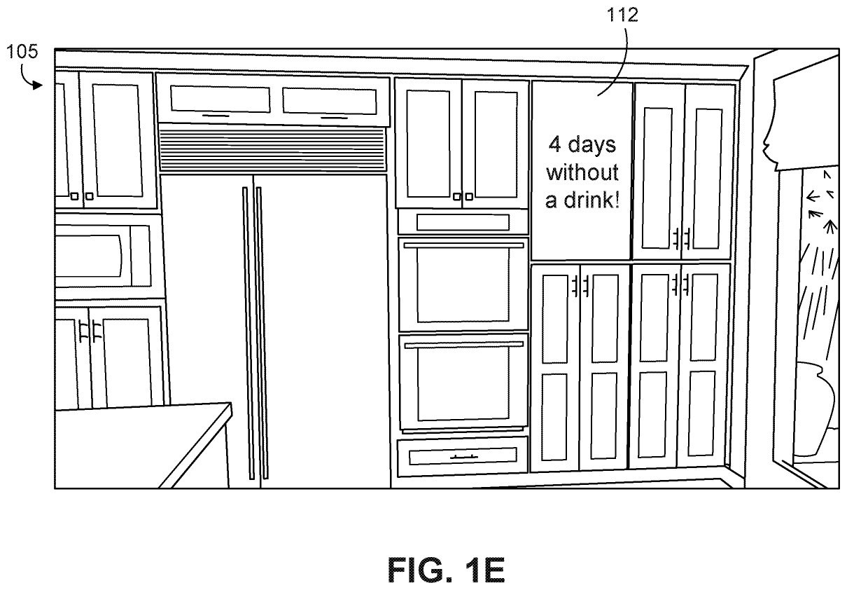

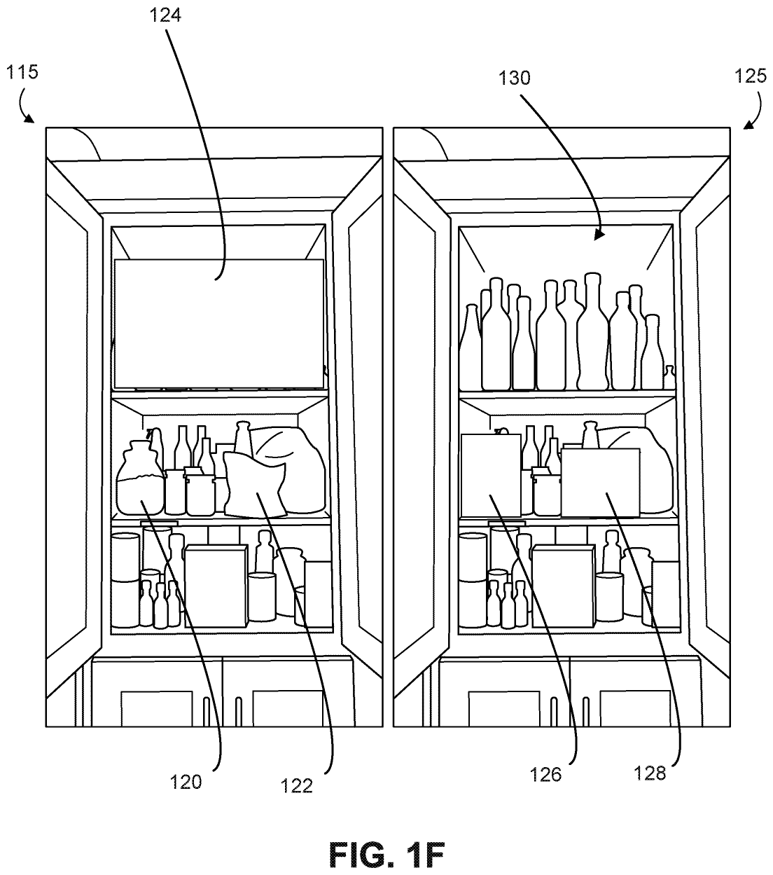

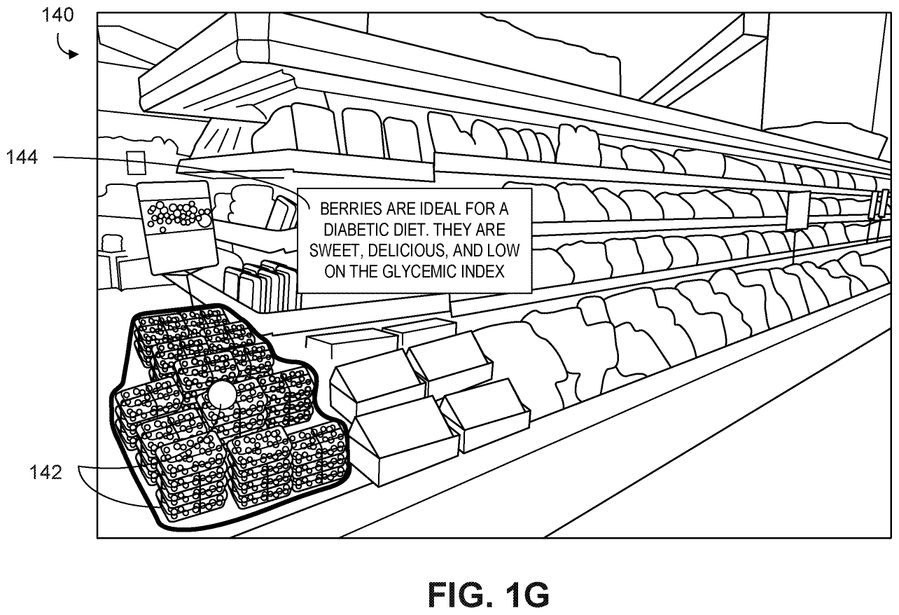

FIG. 1A through FIG. 1G are images illustrating example interventions. In the illustrated examples of FIG. 1A through FIG. 1D, the target behavior can be a user's desire to quit smoking. FIG. 1A illustrates an example package of cigarettes with a generic label 104 illustrated on a visible side of the cigarette package. FIG. 1B illustrates an example progress update intervention 106 indicating progress toward a user's goal projected onto the visible side of the cigarette package 102 that reads “14 days without cigarettes.” FIG. 1C illustrates an example personal message intervention 108 projected onto the visible side of the cigarette package 102 that includes a message from the user's child that reads “I love you mom!” FIG. 1D illustrates an example image intervention 110 projected onto the visible side of the cigarette package 102. In the illustration of FIG. 1D, the image intervention 110 includes a photograph of a parent (e.g., a user of an XR device) holding the parent's young child. In some cases, the example image interventions 106, 108, 110 can be examples of a plurality of available interventions, including other types of interventions described throughout the present disclosure as well as any other type of intervention that the XR system can provide to influence user behavior. In some aspects, the XR system can learn which of the available intervention options is most likely to be effective depending on one or more of the characteristics of the user, context of the intervention, characteristics of the user, or any other relevant factor determined by the XR system and provide the best available intervention to the user. In one illustrative example, the learning system might learn that overlaying a picture of the user's child on a pack of cigarettes (e.g., image intervention 110 shown in FIG. 1D) is effective for users with children in the range of 0-14, but a message indicating progress (e.g., “14 days without a cigarette”) is most effective for users with older children. Instead of using generic assumptions about behaviors and/or responses to interventions, the system can learn what types of intervention are most effective, thus increasing overall system effectiveness.

FIG. 1E through FIG. 1F illustrate additional example interventions. In some cases, obscuring interventions can be used to deter a user from performing an unwanted behavior by obscuring behavioral artifacts (e.g., physical objects associated with the behavior). Image 105 shown in FIG. 1E illustrates cabinets with an intervention 112 obscuring a particular cabinet that the XR system has learned contains alcohol. The intervention 112 also includes a progress update that reads “4 days without a drink.” FIG. 1F illustrates additional obscuring interventions. In the left image 115, a shelf containing alcohol is obscured by intervention 124, while sweet foods 120, 122 remain visible. In the right image 125, the shelf containing alcohol 130 is visible, but interventions 126, 128 are provided to obscure the sweet foods depending on the user's target behavior and/or goals.

FIG. 1G illustrates another example intervention. In the image 140 shown in FIG. 1G, an intervention 142 is shown emphasizing a healthy food option of berries in a market. The intervention 142 can also include an informational message 144 to explain to a user why selecting berries can be beneficial to their health. The intervention 142 shown in FIG. 1G could be provided for multiple different target behaviors. In one illustrative example, the target behavior could be to stop eating processed foods. The XR system could provide the intervention 142 in order to encourage the user to perform a replacement behavior of eating healthy foods. For instance, the target behavior could be to eat low-sugar foods (e.g., to eat more fruits and vegetables). In such a case, the XR system could provide the identical intervention 142 to a user, even if the stated goals are different. In some cases, users with different target behaviors may share one or more common characteristics, and the XR system (or the server) can determine that the same intervention is likely to be effective for the users based on their common characteristics.

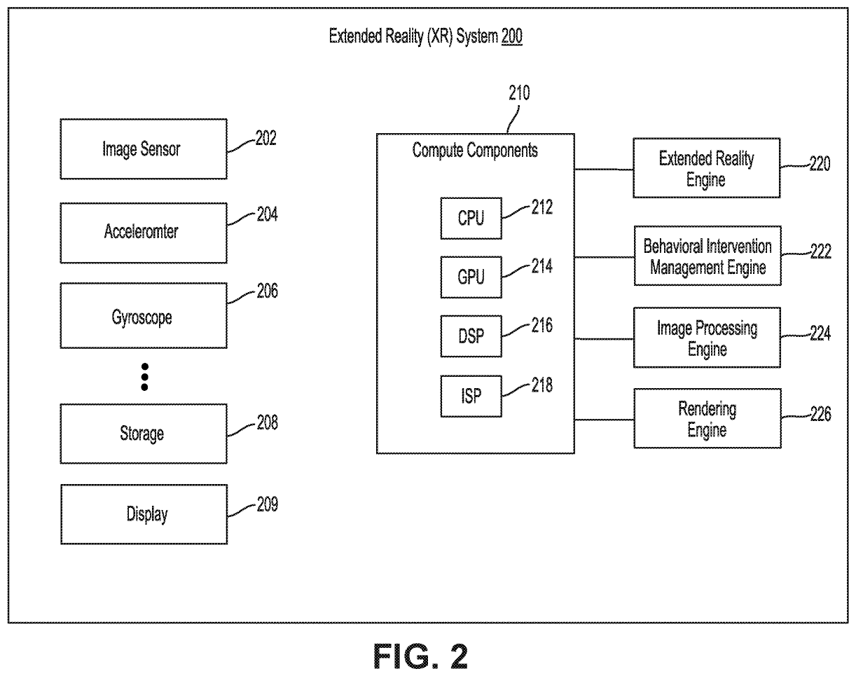

FIG. 2 is a diagram illustrating an example XR system 200, in accordance with some aspects of the disclosure. The XR system 200 can run (or execute) XR applications and implement XR operations. In some examples, the XR system 200 can perform tracking and localization, mapping of the physical world (e.g., a scene), and positioning and rendering of virtual content on a display 209 (e.g., a screen, visible plane/region, and/or other display) as part of an XR experience. For example, the XR system 200 can generate a map (e.g., a three-dimensional (3D) map) of a scene in the physical world, track a pose (e.g., location and position) of the XR system 200 relative to the scene (e.g., relative to the 3D map of the scene), position and/or anchor virtual content in a specific location(s) on the map of the scene, and render the virtual content on display 209 such that the virtual content appears to be at a location in the scene corresponding to the specific location on the map of the scene where the virtual content is positioned and/or anchored. The display 209 can include a glass, a screen, a lens, a projector, and/or other display mechanism that allows a user to see the real-world environment and also allows XR content to be overlaid, overlapped, blended with, or otherwise displayed thereon. As described below, the XR system 200 can perform attention tracking.

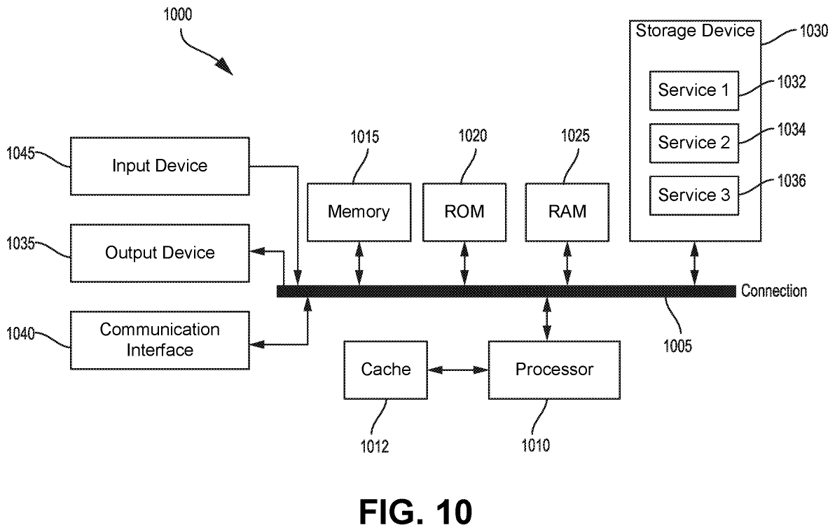

In the example of FIG. 2, the XR system 200 includes one or more image sensors 202, an accelerometer 204, a gyroscope 206, storage 208, display 209, compute components 210, an XR engine 220, a behavioral intervention management engine 222, an image processing engine 224, and a rendering engine 226. It should be noted that the components 202 through 226 shown in FIG. 2 are non-limiting examples provided for illustrative and explanation purposes, and other examples can include more, fewer, or different components than those shown in FIG. 2. For example, in some cases, the XR system 200 can include one or more other sensors (e.g., one or more inertial measurement units (IMUs), radars, light detection and ranging (LIDAR) sensors, audio sensors, etc.), one or more display devices, one more other processing engines, one or more eye tracking sensors, one or more speakers (e.g., for providing auditory feedback or other audio output), one or more microphones (e.g., for capturing auditory input and/or other sound or audio), one or more other hardware components, and/or one or more other software and/or hardware components that are not shown in FIG. 2. An example architecture and example hardware components that can be implemented by the XR system 200 are further described below with respect to computing system 1000 shown in FIG. 10.

Moreover, for simplicity and explanation purposes, the one or more image sensors 202 will be referenced herein as an image sensor 202 (e.g., in singular form). However, one of ordinary skill in the art will recognize that the XR system 200 can include a single image sensor or multiple image sensors. Also, references to any of the components (e.g., 202-226) of the XR system 200 in the singular or plural form should not be interpreted as limiting the number of such components implemented by the XR system 200 to one or more than one. For example, references to an accelerometer 204 in the singular form should not be interpreted as limiting the number of accelerometers implemented by the XR system 200 to one. One of ordinary skill in the art will recognize that, for any of the components 202 through 226 shown in FIG. 2, the XR system 200 can include only one of such component(s) or more than one of such component(s).

The XR system 200 can be part of, or implemented by, a single computing device or multiple computing devices. In some examples, the XR system 200 can be part of an electronic device (or devices) such as a camera system (e.g., a digital camera, an IP camera, a video camera, a security camera, etc.), a telephone system (e.g., a smartphone, a cellular telephone, a conferencing system, etc.), a desktop computer, a laptop or notebook computer, a tablet computer, a set-top box, a smart television, a display device, a gaming console, a video streaming device, an IoT (Internet-of-Things) device, a smart wearable device (e.g., a head-mounted display (HMD), smart glasses, etc.), or any other suitable electronic device(s).

In some implementations, the one or more image sensors 202, the accelerometer 204, the gyroscope 206, storage 208, display 209, compute components 210, XR engine 220, behavioral intervention management engine 222, image processing engine 224, rendering engine 226, and any other component now shown in FIG. 2 (e.g., microphone(s), speaker(s), eye tracking sensor(s), etc.) can be part of the same computing device. For example, in some cases, the one or more image sensors 202, the accelerometer 204, the gyroscope 206, storage 208, compute components 210, XR engine 220, behavioral intervention management engine 222, image processing engine 224, and rendering engine 226 can be integrated into a smartphone, laptop, tablet computer, smart wearable device, gaming system, and/or any other computing device. However, in some implementations, the one or more image sensors 202, the accelerometer 204, the gyroscope 206, storage 208, compute components 210, XR engine 220, behavioral intervention management engine 222, image processing engine 224, and rendering engine 226 can be part of two or more separate computing devices. For example, in some cases, some of the components 202 through 226 can be part of, or implemented by, one computing device and the remaining components can be part of, or implemented by, one or more other computing devices.

The image sensor 202 can include any image and/or video sensors or capturing devices, such as a digital camera sensor, a video camera sensor, a smartphone camera sensor, an image/video capture device on an electronic apparatus such as a television or computer, a camera, etc. In some cases, the image sensor 202 can be part of a camera or computing device such as an XR device (e.g., an HMD, smart glasses, etc.), a digital camera, a smartphone, a smart television, a game system, etc. In some examples, the image sensor 202 can be part of a multiple-camera assembly, such as a dual-camera assembly. The image sensor 202 can capture image and/or video content (e.g., raw image and/or video data), which can then be processed by the compute components 210, the XR engine 220, the behavioral intervention management engine 222, the image processing engine 224, and/or the rendering engine 226 as described herein.

In some examples, the image sensor 202 can capture image data and generate frames based on the image data and/or provide the image data or frames to the XR engine 220, the behavioral intervention management engine 222, the image processing engine 224 and/or the rendering engine 226 for processing. A frame can include a video frame of a video sequence or a still image. A frame can include a pixel array representing a scene. For example, a frame can be a red-green-blue (RGB) frame having red, green, and blue color components per pixel; a luma, chroma-red, chroma-blue (YCbCr) frame having a luma component and two chroma (color) components (chroma-red and chroma-blue) per pixel; or any other suitable type of color or monochrome picture.

The accelerometer 204 can detect acceleration by the XR system 200 and generate acceleration measurements based on the detected acceleration. The gyroscope 206 can detect and measure the orientation and angular velocity of the XR system 200. For example, the gyroscope 206 can be used to measure the pitch, roll, and yaw of the XR system 200. In some examples, the image sensor 202 and/or the XR engine 220 can use measurements obtained by the accelerometer 204 and the gyroscope 206 to calculate the pose of the XR system 200. As previously noted, in other examples, the XR system 200 can also include other sensors, such as a magnetometer, a machine vision sensor, a smart scene sensor, a speech recognition sensor, an impact sensor, a shock sensor, a position sensor, a tilt sensor, etc.

The storage 208 can be any storage device(s) for storing data. Moreover, the storage 208 can store data from any of the components of the XR system 200. For example, the storage 208 can store data from the image sensor 202 (e.g., image or video data), data from the accelerometer 204 (e.g., measurements), data from the gyroscope 206 (e.g., measurements), data from the compute components 210 (e.g., processing parameters, preferences, virtual content, rendering content, scene maps, tracking and localization data, object detection data, privacy data, XR application data, face recognition data, occlusion data, etc.), data from the XR engine 220, data from the behavioral intervention management engine 222, data from the image processing engine 224, and/or data from the rendering engine 226 (e.g., output frames). In some examples, the storage 208 can include a buffer for storing frames for processing by the compute components 210.

The one or more compute components 210 can include a central processing unit (CPU) 212, a graphics processing unit (GPU) 214, a digital signal processor (DSP) 216, and/or an image signal processor (ISP) 218. The compute components 210 can perform various operations such as image enhancement, computer vision, graphics rendering, XR (e.g., tracking, localization, pose estimation, mapping, content anchoring, content rendering, etc.), image/video processing, sensor processing, recognition (e.g., text recognition, facial recognition, object recognition, feature recognition, tracking or pattern recognition, scene recognition, occlusion detection, etc.), machine learning, filtering, and any of the various operations described herein. In this example, the compute components 210 implement the XR engine 220, the behavioral intervention management engine 222, the image processing engine 224, and the rendering engine 226. In other examples, the compute components 210 can also implement one or more other processing engines.

The operations for the XR engine 220, the behavioral intervention management engine 222, the image processing engine 224, and the rendering engine 226 (and any image processing engines) can be implemented by any of the compute components 210. In one illustrative example, the operations of the rendering engine 226 can be implemented by the GPU 214, and the operations of the XR engine 220, the behavioral intervention management engine 222, and the image processing engine 224 can be implemented by the CPU 212, the DSP 216, and/or the ISP 218. In some cases, the compute components 210 can include other electronic circuits or hardware, computer software, firmware, or any combination thereof, to perform any of the various operations described herein.

In some examples, the XR engine 220 can perform XR operations based on data from the image sensor 202, the accelerometer 204, the gyroscope 206, and/or one or more sensors on the XR system 200, such as one or more IMUs, radars, etc. In some examples, the XR engine 220 can perform tracking, localization, pose estimation, mapping, content anchoring operations and/or any other XR operations/functionalities.

The behavioral intervention management engine 222 can perform behavior detection and generate behavior interventions. In some cases, when the behavioral intervention management engine 222 determines (e.g., with a likelihood algorithm) that an unwanted behavior is likely to occur (or that a wanted behavior is unlikely to occur) and/or that a healthy behavior is likely to occur, the behavioral intervention management engine 222 can generate and can perform an intervention. In some cases, the behavioral intervention management engine 222 can include one or more learning systems that can improve upon the efficacy of interventions and/or the accuracy of behavior detection. For example, the behavioral intervention management engine 222 can determine whether a performed intervention successfully influenced a user's behavior (e.g., to perform a healthy behavior, to not perform an unhealthy behavior, etc.). In some cases, based on whether the intervention was successful or not, the behavioral intervention management engine 222 can update the efficacy of the intervention in an intervention library.

In some cases, the behavioral intervention management engine 222 can also improve upon the accuracy of behavior predictions. For example, if the behavioral intervention management engine 222 determines that an unwanted behavior is unlikely to occur and determines not to generate an intervention, the user may still perform the unwanted behavior. The behavior management engine 222 can use the inaccurate behavior prediction to update a behavior likelihood algorithm. In one illustrative example, the behavioral intervention management system can update weights associated with behavioral information (e.g., behavioral triggers, pre-behaviors, and/or behavioral artifacts) used to determine the likelihood of the behavior occurring (or not occurring). In another illustrative example, the behavioral intervention management engine 222 can determine whether additional behavioral information specific to the user was omitted from the likelihood determination. For example, the behavioral intervention management engine 222 can determine that a particular user has a unique behavioral trigger and include the unique behavioral trigger when determining subsequent likelihood determinations.

In some aspects, the behavioral intervention management engine 222 can communicate with a server to send indications of the effectiveness of interventions on the user's behavior and/or indications of accuracy of determining the likelihood of the user performing (or not performing) the target behavior. In some cases, the behavioral intervention management engine 222 can also send characteristics (e.g., gender, age, family status) of the user, contexts of the behavior and/or intervention (e.g., locale, combinations of technologies/sensors in use), behavioral information (e.g., behavioral triggers, pre-behaviors) associated with determining the likelihood of the behavior and/or determining which intervention to generate for the user to the server, any combination thereof, and/or other information. In some cases, the behavioral intervention management engine 222 can receive interventions and/or behavior prediction models from a server. In some cases, the received interventions and/or behavior prediction models can be based on characteristics, contexts, and/or behavioral information determined from multiple users. In some cases, the behavioral intervention management engine 222 can receive interventions and/or behavior prediction models from the server that are associated with characteristics and/or contexts shared between the user and a subset of other users.

The image processing engine 224 can perform one or more image processing operations. In some examples, the image processing engine 224 can perform image processing operations based on data from the image sensor 202. The rendering engine 226 can obtain image data generated and/or processed by the compute components 210, the image sensor 202, the XR engine 220, and/or the image processing engine 224 and render video and/or image frames for presentation on a display device.

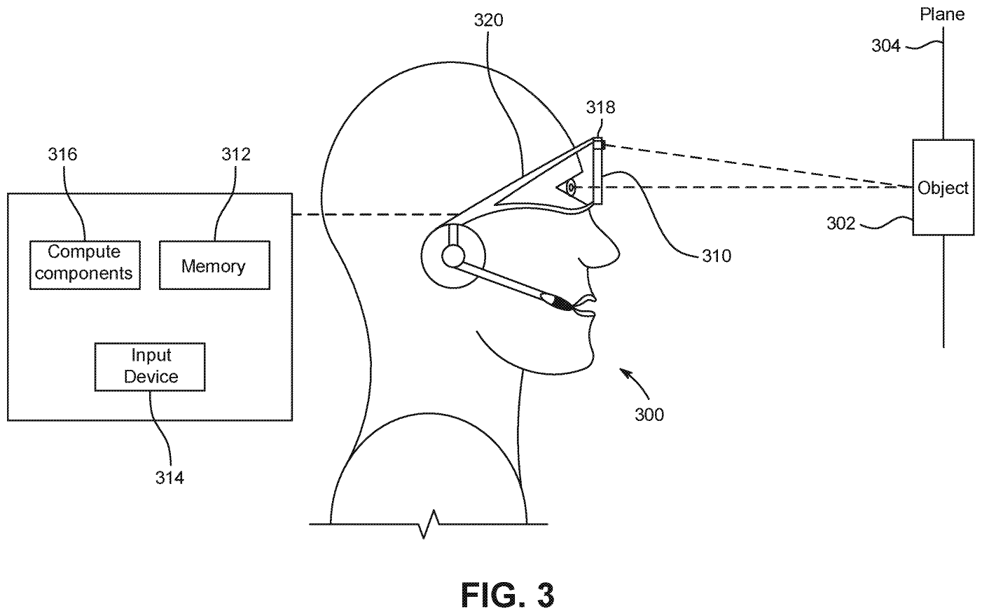

FIG. 3 is a diagram illustrating an example of an XR system 320 being worn by a user 300. The XR system 320 can include some or all of the same components as the XR system 200 shown in FIG. 2 and described above, and can perform some or all of the same functions as the XR system 200. While the XR system 320 is shown in FIG. 2 as AR glasses, the XR system 320 can include any suitable type of XR device, such as an HMD or other XR devices. The XR system 320 is described as an optical see-through AR device, which allows the user 300 to view the real world while wearing the XR system 320. For example, the user 300 can view an object 302 in a real-world environment on a plane 304 at a distance from the user 300. The XR system 320 has an image sensor 318 and a display 310 (e.g., a glass, a screen, a lens, or other display) that allows the user 300 to see the real-world environment and also allows AR content to be displayed thereon. The image sensor 318 can be similar or the same as the image sensor 202 shown in FIG. 2. While one image sensor 318 and one display 310 are shown in FIG. 3, the XR system 320 can include multiple cameras and/or multiple displays (e.g., a display for the right eye and a display for the left eye) in some implementations. AR content (e.g., an image, a video, a graphic, a virtual or AR object, or other AR content) can be projected or otherwise displayed on the display 310. In one example, the AR content can include an augmented version of the object 302. In another example, the AR content can include additional AR content that is related to the object 302 or related to one or more other objects in the real-world environment.

As shown in FIG. 3, the XR system 320 can include, or can be in wired or wireless communication with, compute components 316 and a memory 312. The compute components 316 and the memory 312 can store and execute instructions used to perform the techniques described herein. In implementations where the XR system 320 is in communication (wired or wirelessly) with the memory 312 and the compute components 316, a device housing the memory 312 and the compute components 316 may be a computing device, such as a desktop computer, a laptop computer, a mobile phone, a tablet, a game console, or other suitable device. The XR system 320 also includes or is in communication with (wired or wirelessly) an input device 314. The input device 314 can include any suitable input device, such as a touchscreen, a pen or other pointer device, a keyboard, a mouse a button or key, a microphone for receiving voice commands, a gesture input device for receiving gesture commands, any combination thereof, and/or other input device. In some cases, the image sensor 318 can capture images that can be processed for interpreting gesture commands.

The image sensor 318 can capture color images (e.g., images having red-green-blue (RGB) color components, images having luma (Y) and chroma (C) color components such as YCbCr images, or other color images) and/or grayscale images. As noted above, in some cases, the XR system 320 can include multiple cameras, such as dual front cameras and/or one or more front and one or more rear-facing cameras, which may also incorporate various sensors. In some cases, image sensor 318 (and/or other cameras of the XR system 320) can capture still images and/or videos that include multiple video frames (or images). In some cases, image data received by the image sensor 318 (and/or other cameras) can be in a raw uncompressed format, and may be compressed and/or otherwise processed (e.g., by an image signal processor (ISP) or other processor of the XR system 320) prior to being further processed and/or stored in the memory 312. In some cases, image compression may be performed by the compute components 316 using lossless or lossy compression techniques (e.g., any suitable video or image compression technique).

In some cases, the image sensor 318 (and/or other camera of the XR system 320) can be configured to also capture depth information. For example, in some implementations, the image sensor 318 (and/or other camera) can include an RGB-depth (RGB-D) camera. In some cases, the XR system 320 can include one or more depth sensors (not shown) that are separate from the image sensor 318 (and/or other camera) and that can capture depth information. For instance, such a depth sensor can obtain depth information independently from the image sensor 318. In some examples, a depth sensor can be physically installed in a same general location the image sensor 318, but may operate at a different frequency or frame rate from the image sensor 318. In some examples, a depth sensor can take the form of a light source that can project a structured or textured light pattern, which may include one or more narrow bands of light, onto one or more objects in a scene. Depth information can then be obtained by exploiting geometrical distortions of the projected pattern caused by the surface shape of the object. In one example, depth information may be obtained from stereo sensors such as a combination of an infra-red structured light projector and an infra-red camera registered to a camera (e.g., an RGB camera).

In some implementations, the XR system 320 includes one or more sensors. The one or more sensors can include one or more accelerometers (e.g., accelerometer 204 shown in FIG. 2), one or more gyroscopes (e.g., gyroscope 206 shown in FIG. 2), and/or other sensors. The one or more sensors can provide velocity, orientation, and/or other position-related information to the compute components 316. In some cases, the one or more sensors can include at least one inertial measurement unit (IMU). An IMU is an electronic device that measures the specific force, angular rate, and/or the orientation of the XR system 320, using a combination of one or more accelerometers, one or more gyroscopes, and/or one or more magnetometers. In some examples, the one or more sensors can output measured information associated with the capture of an image captured by the image sensor 318 (and/or other camera of the XR system 320) and/or depth information obtained using one or more depth sensors of the XR system 320.

The output of one or more sensors (e.g., one or more IMUs) can be used by the compute components 316 to determine a pose of the XR system 320 (also referred to as the head pose) and/or the pose of the image sensor 318 (or other camera of the XR system 320). In some cases, the pose of the XR system 320 and the pose of the image sensor 318 (or other camera) can be the same. The pose of image sensor 318 refers to the position and orientation of the image sensor 318 relative to a frame of reference (e.g., with respect to the object 302). In some implementations, the camera pose can be determined for 6-Degrees Of Freedom (6DOF), which refers to three translational components (e.g., which can be given by X (horizontal), Y (vertical), and Z (depth) coordinates relative to a frame of reference, such as the image plane) and three angular components (e.g. roll, pitch, and yaw relative to the same frame of reference).

In some aspects, the pose of image sensor 318 and/or the XR system 320 can be determined and/or tracked by the compute components 316 using a visual tracking solution based on images captured by the image sensor 318 (and/or other camera of the XR system 320). In some examples, the compute components 316 can perform tracking using computer vision-based tracking, model-based tracking, and/or simultaneous localization and mapping (SLAM) techniques. For instance, the compute components 316 can perform SLAM or can be in communication (wired or wireless) with a SLAM engine (now shown). SLAM refers to a class of techniques where a map of an environment (e.g., a map of an environment being modeled by XR system 320) is created while simultaneously tracking the pose of a camera (e.g., image sensor 318) and/or the XR system 320 relative to that map. The map can be referred to as a SLAM map, and can be three-dimensional (3D). The SLAM techniques can be performed using color or grayscale image data captured by the image sensor 318 (and/or other camera of the XR system 320), and can be used to generate estimates of 6DOF pose measurements of the image sensor 318 and/or the XR system 320. Such a SLAM technique configured to perform 6DOF tracking can be referred to as 6DOF SLAM. In some cases, the output of one or more sensors can be used to estimate, correct, and/or otherwise adjust the estimated pose.

In some cases, the 6DOF SLAM (e.g., 6DOF tracking) can associate features observed from certain input images from the image sensor 318 (and/or other camera) to the SLAM map. 6DOF SLAM can use feature point associations from an input image to determine the pose (position and orientation) of the image sensor 318 and/or XR system 320 for the input image. 6DOF mapping can also be performed to update the SLAM Map. In some cases, the SLAM map maintained using the 6DOF SLAM can contain 3D feature points triangulated from two or more images. For example, key frames can be selected from input images or a video stream to represent an observed scene. For every key frame, a respective 6DOF camera pose associated with the image can be determined. The pose of the image sensor 318 and/or the XR system 320 can be determined by projecting features from the 3D SLAM map into an image or video frame and updating the camera pose from verified 2D-3D correspondences.

In one illustrative example, the compute components 316 can extract feature points from every input image or from each key frame. A feature point (also referred to as a registration point) as used herein is a distinctive or identifiable part of an image, such as a part of a hand, an edge of a table, among others. Features extracted from a captured image can represent distinct feature points along three-dimensional space (e.g., coordinates on X, Y, and Z-axes), and every feature point can have an associated feature location. The features points in key frames either match (are the same or correspond to) or fail to match the features points of previously-captured input images or key frames. Feature detection can be used to detect the feature points. Feature detection can include an image processing operation used to examine one or more pixels of an image to determine whether a feature exists at a particular pixel. Feature detection can be used to process an entire captured image or certain portions of an image. For each image or key frame, once features have been detected, a local image patch around the feature can be extracted. Features may be extracted using any suitable technique, such as Scale Invariant Feature Transform (SIFT) (which localizes features and generates their descriptions), Speed Up Robust Features (SURF), Gradient Location-Orientation histogram (GLOH), Normalized Cross Correlation (NCC), or other suitable technique.

In some examples, AR (or virtual) objects can be registered or anchored to (e.g., positioned relative to) the detected features points in a scene. For example, the user 300 can be looking at a restaurant across the street from where the user 300 is standing. In response to identifying the restaurant and AR content associated with the restaurant, the compute components 316 can generate an AR object that provides information related to the restaurant. The compute components 316 can also detect feature points from a portion of an image that includes a sign on the restaurant, and can register the AR object to the feature points of the sign so that the AR object is displayed relative to the sign (e.g., above the sign so that it is easily identifiable by the user 300 as relating to that restaurant). In addition, interventions as described herein (e.g., interventions 106, 108, 110, 112, 124, 126, 128, 142 shown in FIG. 1B through FIG. 1G)

The XR system 320 can generate and display various AR objects for viewing by the user 300. For example, the XR system 320 can generate and display a virtual interface, such as a virtual keyboard, as an AR object for the user 300 to enter text and/or other characters as needed. The virtual interface can be registered to one or more physical objects in the real world. However, in many cases, there can be a lack of real-world objects with distinctive features that can be used as reference for registration purposes. For example, if a user is staring at a blank whiteboard, the whiteboard may not have any distinctive features to which the virtual keyboard can be registered. Outdoor environments may provide even less distinctive points that can be used for registering a virtual interface, for example based on the lack of points in the real world, distinctive objects being further away in the real world than when a user is indoors, the existence of many moving points in the real world, points at a distance, among others.

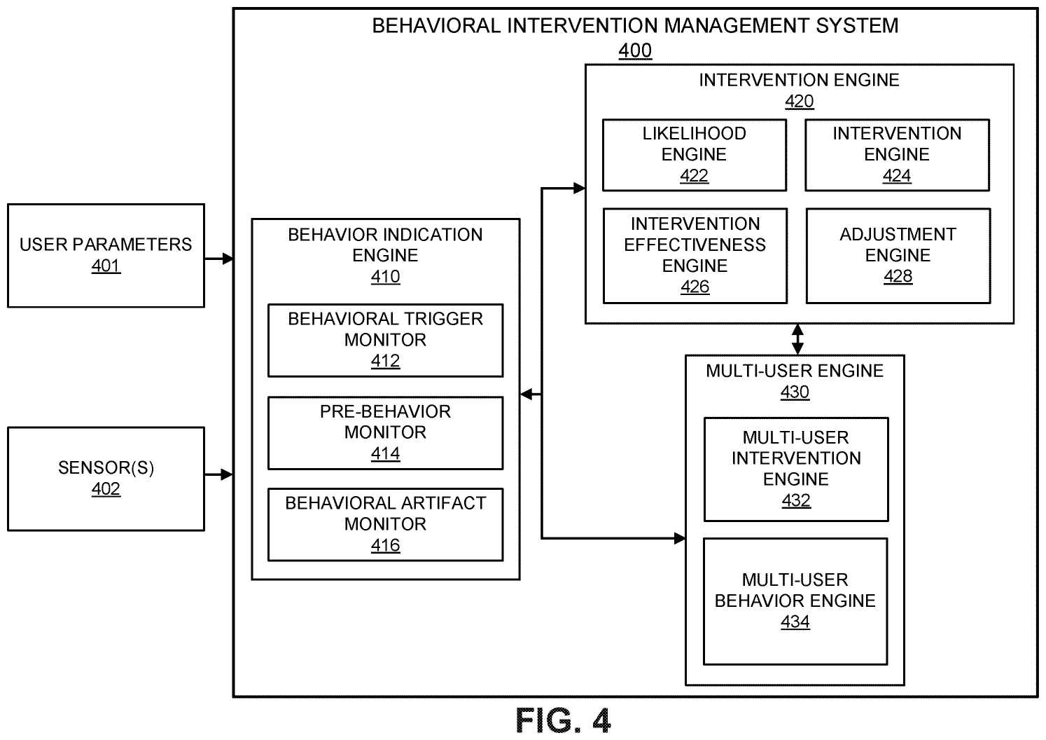

FIG. 4 illustrates a block diagram of a behavioral intervention management system 400 for detecting behaviors and generating interventions. The behavioral intervention management system 400 can obtain user parameters 401 which can include goals and/or plans for behavior change. Examples of user parameters 401 are provided below. The behavioral intervention management system 400 can detect when an unwanted behavior is likely to occur or when an intervention for a desired behavior is more likely to be successful. Based on the determination, the behavioral intervention management system 400 can generate an intervention to influence the user's behavior to discourage the user from performing the unwanted behavior or encourage the user to perform a wanted behavior.

In this illustrative example of FIG. 4, the behavioral intervention management system 400 includes a behavior indication engine 410, an intervention engine 420, and a multi-user engine 430. It should be noted that the components 410 through 430 shown in FIG. 4 are non-limited examples provided for illustrative and explanation purposes and other examples can include more, fewer, or different components than those shown in FIG. 4 without departing from the scope of the present disclosure. In one illustrative example, one or more of the components of the behavioral intervention management system can be included in, or can include, the behavioral intervention management engine 222 shown in FIG. 2.

The behavioral intervention management system 400 can be part of, or implemented by, a single computing device or multiple computing devices. In some implementations, the behavior indication engine 410, the intervention engine 420, and the multi-user engine 430 can be part of the same computing device. For example, the behavioral intervention management system 400 can be part of an electronic device (or devices) such as a camera system (e.g., a digital camera, an IP camera, a video camera, a security camera, etc.), a telephone system (e.g., a smartphone, a cellular telephone, a conferencing system, etc.), a desktop computer, a laptop or notebook computer, a tablet computer, a set-top box, a smart television, a display device, a gaming console, a video streaming device, an IoT device, a smart wearable device (e.g., a HMD, smart glasses, etc.), or any other suitable electronic device(s). However, in some implementations, the behavior indication engine 410, the intervention engine 420, and the multi-user engine 430 can be part of two or more separate computing devices. For example, in some cases, some of the components 410 through 430 can be part of, or implemented by, one computing device and the remaining components can be part of, or implemented by, one or more other computing devices. In one illustrative example, the multi-user engine 430 can be part of or implemented on a server that receives behavior accuracy indications and intervention efficacy indications from multiple different user devices and the behavior indication engine 410 and intervention engine 420 can be implemented on a user device.

The behavioral intervention management system 400 can obtain one or more user parameters 401. The user parameters 401 can include, but are not limited to, user goals and plans, user characteristics, user behaviors, users motivations for change (e.g., a desire to be able to play with grandchildren), one or more health conditions (e.g., high blood pressure, weight issues, diabetes, heart disease, hypertension, etc.), one or more dietary restrictions of the user (e.g., lactose intolerant, high fiber diet, low calorie diet, etc.), one or more physical capabilities of the user, environmental factors that may support or limit certain behaviors, and any other information that could potentially be relevant in anticipating the user's behaviors and/or successfully generating interventions to influence the user's behaviors. In some cases, user parameters 401 can be obtained directly from a user, such as a user input through a user interface. In some cases, the user parameters 401 can be input from one or more sources in addition to or as an alternative to user entered parameters. In one illustrative example, user parameters 401 can be obtained during an onboarding process, such as when the user purchases an XR system (e.g., XR system 200 shown in FIG. 2). In another illustrative example, user parameters 401 can be entered by a physician or an insurance company. In another illustrative example, user parameters 401 can be obtained from one or more other devices belonging to a user and/or one or more services in which the user participates, such as profile settings, health and fitness tracking data, or the like. In some examples, standard user parameters can be specified for certain types of users. For instance, parameters can be defined for users with certain health conditions (e.g., users with diabetes, heart disease, hypertension, COPD, etc.), so that multiple users with similar health conditions can be associated with similar interventions.

The behavioral intervention management system 400 can also obtain data from one or more sensors 402 to enable detection of user behavioral information. For example, the one or more sensors can include one or more image sensors (e.g., image sensor 202 shown in FIG. 2), microphones, accelerometers, (e.g., accelerometer 204), location sensor (e.g., GPS sensor), eye tracking sensors, contact tracing sensors (e.g., Bluetooth™ sensors, etc.) which can detect when a person is in close proximity with another person or a group of people, or any other sensors that can be used to detect a user's behavior and/or environment (e.g., to identify that person A is with person B at a restaurant ordering food). In some cases, one or more of the sensors 402 can be included in a same device (e.g., XR system 200) as the behavioral intervention management system 400. In some cases, one or more of the sensors 402 can be included in other devices, such as a fitness tracker, a mobile telephone, IoT devices, or the like.

The behavior indication engine 410 can obtain data from the one or more sensors 402 in order to identify behavioral information associated with the user. In the illustrative example of FIG. 4, the behavior indication engine 410 includes a behavioral trigger monitor 412, a pre-behavior monitor 414, and a behavioral artifact monitor 416. Each of the behavioral trigger monitor 412, the pre-behavior monitor 414, and the behavioral artifact monitor 416 can process the data from one or more of the sensors 402 to detect corresponding behavior information.

The behavioral trigger monitor 412 can process data from the one or more sensors to identify behavioral triggers (e.g., stress level, location, environment, time, nearby people, nearby objects, etc.). For example, the behavioral trigger monitor 412 can process data from one or more of a heart rate monitor, a galvanic skin response sensor, a blood pressure sensor to detect a stress response in the user, and/or other sensors.

The pre-behavior monitor 414 can process data from the one or more sensors 402 to identify pre-behaviors, which can be behaviors that indicate that the user is likely to engage (or likely not to engage) or has decided to engage (or not engage) in a behavior. For example, the pre-behavior monitor 414 can obtain image sensor data that shows the user is walking toward a cabinet with alcohol, reaching for a pack of cigarettes, or performing another type of pre-behavior indicating the user is likely to engage/not engage (or has engaged/not engaged) in a particular behavior.

The behavioral artifact monitor 416 can process data from the one or more sensors to identify behavioral artifacts. For example, the behavioral artifact monitor 416 and/or the behavior indication engine 410 can perform feature detection (e.g., by an ML model, such as one or more neural networks) on images received from an image sensor of the sensors 402 and assign classes (e.g., table, child, car, cabinet, bottle, cigarettes, etc.) to different features detected in the images. In one illustrative example, the behavioral artifact monitor 416 can process the assigned classes to determine whether any of the images contain behavioral artifacts (e.g., a pack of cigarettes, an ice cream container).

The behavior indication engine 410 can provide behavioral information from one or more of the behavioral trigger monitor 412, the pre-behavior monitor 414, and the behavioral artifact monitor 416 to the intervention engine 420. In some cases, the behavior indication engine 410 can also optionally provide behavioral information from one or more of the behavioral trigger monitor 412, the pre-behavior monitor 414, and the behavioral artifact monitor 416 to a multi-user engine 430.

As illustrated, intervention engine 420 includes a likelihood engine 422, an intervention engine 424, an intervention effectiveness engine 426, and an adjustment engine 428. The intervention engine 420 can obtain behavioral information from the behavior indication engine 410 and process the behavioral information. The intervention engine 420 can determine whether target behaviors are likely to occur (or not occur) and generate interventions to influence the user away from undesired behaviors (or toward desired behaviors) based on the likelihood determination.

Likelihood engine 422 can determine the likelihood of a behavior occurring or not occurring. In one example implementation, the likelihood engine 422 can determine the likelihood of the behavior occurring based on applying different weights to individual components of the behavioral information obtained from the behavior indication engine 410. For example, the likelihood engine may assign a high weighting to pre-behaviors because they indicate an intent by the user to perform a behavior, while behavioral artifacts coming into view may be assigned a low weighting because a particular user's behavior is not strongly affected by seeing behavioral artifacts. In some cases, the likelihood of a behavior occurring (or not occurring) determined by the likelihood engine 422 can be compared to a threshold. In some cases, if the likelihood exceeds the threshold, the intervention engine 420 can generate an intervention for the user to attempt to influence the user's behavior. In some cases, the behavioral intervention management system 400 can monitor the user's behavior after the prediction to determine whether the prediction from the likelihood engine 422 was accurate. In some implementations, the likelihood engine 422 can be trained during a training period before any interventions are applied by the behavioral intervention management system 400. In some aspects, the likelihood engine 422 can be implemented by a ML model, such as one or more neural networks. The likelihood engine 422 can continuously monitor the behavioral information from the behavior indication engine 410 to determine that a behavior is likely to occur (or not occur) before it happens.

Intervention engine 424 can obtain the likelihood determined by the likelihood engine 422. If the intervention engine 424 determines that the likelihood indicates an intervention is required (e.g., the likelihood excepts a threshold), the intervention engine 424 can generate an intervention. In some cases, the intervention engine 424 can include an intervention library to select from to influence the user's behavior. For example, the intervention library can include any of the interventions described herein, including interventions 106, 108, 110, 112, 124, 126, 128 shown in FIG. 1B through FIG. 1G, audio interventions, contacting a support person, or the like.

Intervention effectiveness can be situational. For example, for a particular individual, an intervention might be effective in some situations (e.g. in the morning or when the person is alone) and ineffective in other situations (e.g. in the evening or around other people). The intervention effectiveness engine 426 can determine whether, after the intervention engine 420 generates an intervention, an effectiveness of the intervention. In one illustrative example, the intervention effectiveness engine 426 can monitor the behavior of a user after an intervention is generated to determine whether the intervention effectively influenced the user's behavior. For instance, if the user engages in an unwanted behavior after the intervention, the intervention effectiveness engine 426 can determine that the intervention was ineffective for the particular user. In some cases, the intervention effectiveness engine 426 can determine that an intervention was partially effective for a user, such as when the user engages in a behavior, but to a reduced degree to previous times the user engaged in the same behavior. In one illustrative example, the user may smoke one cigarette when normally they smoke two.

Adjustment engine 428 can determine one or more adjustments for the intervention engine 420 based on the accuracy of behavior prediction by likelihood engine 422 and/or the effectiveness of interventions generated by the intervention engine 424. For example, the adjustment engine 428 can adjust one or more parameters of the intervention engine 424 to increase the likelihood of effective interventions being generated and decrease the likelihood of ineffective interventions being generated. Similarly, the adjustment engine 428 can adjust one or more parameters of the likelihood engine (e.g., weightings applied to components of behavioral information) based on whether the likelihood engine accuracy predicated a behavior or not.

Multi-user engine 430 can obtain indications of intervention effectiveness and/or accuracy of behavior predictions from intervention engine 420. In the illustrated example of FIG. 4, the multi-user engine 430 includes a multi-user intervention engine 432 and a multi-user behavior engine 434. In some cases, the multi-user engine 430 can obtain characteristics of the user (e.g., user parameters 401), and/or contextual information associated with the intervention. The contextual information can include, without limitation, a time of day, one or more actions by the user prior to the intervention, the behavioral information associated with the user and/or the intervention, a location and/or environment associated with the location of the user (and/or the XR device) before and/or during the intervention, a proximity of the user to one or more individuals, and the combinations of technologies/sensors in use by the user at the time of the intervention. For example, the environment associated with the location of the user can include whether or not there are stairs a person can take, is the user in a kitchen, is there space available for the user to exercise, weather at the user's location (e.g., hot, cold, raining, windy, etc.), presence of a behavioral artifact (e.g., cigarettes, running shoes), etc. The characteristics can include, without limitation, gender, age of the user, family status, target behavior, severity of problem, country/culture, personality type (e.g., fear response, disgust response), one or more health conditions (e.g., diabetes, heart arrythmia, etc.), one or more dietary restrictions (e.g., low calorie diet, vegetarian, etc.), one or more physical capabilities (e.g., weak or injured back, paralysis, etc.), and/or other characteristics.

The multi-user intervention engine 432 can obtain indications of effectiveness of interventions for multiple users. The multi-user intervention engine 432 can process the indications of effectiveness of interventions, the characteristics of the users associated with the interventions, and/or the context of the interventions to generate a multi-user intervention library. In some cases, the interventions in the intervention library can include a score based on effectiveness. In some cases, the interventions can include a separate score for each type or category of intervention and for contexts associated with the interventions, as well as any combinations thereof. In some cases, the multi-user intervention engine 432 can be implemented by a ML model, such as one or more neural networks. In some cases, the multi-user intervention engine 432 can send indications of effectiveness stored in the multi-user intervention that correspond to characteristics of a user and/or contexts frequently experienced by a user to intervention engine 420 and/or adjustment engine 428 in order to provide interventions that are likely to be effective for the user based on demonstrated effectiveness for similar users in similar contexts. In one illustrative example, the multi-user intervention engine may determine that overlaying a picture of a user holding their child on a pack of cigarettes (e.g., intervention 110 shown in FIG. 1D) is effective for users with children age fourteen and younger, but a message indicating progress (e.g., intervention 106 shown in FIG. 1B, intervention 112 shown in FIG. 1E) is more effective for users with children over the age of fourteen.

The multi-user behavior engine 434 can similarly generate a multi-user behavior library. In some cases, the multi-user behavior library can include predictive information (e.g., behavioral triggers, pre-behaviors, and/or behavioral artifacts) that can be associated with user characteristics and/or contexts frequently experienced by a user. In addition to or as an alternative to including predictive information in the multi-user behavior library, the multi-user behavior engine 434 can include behavior likelihood determination information that can be associated with user characteristics and/or contexts frequently experienced by a user. In one illustrative example, the likelihood determination information can include weightings for different types of behavioral information that are most likely to accurately predict behavior based on characteristics and/or frequently experienced contexts. In some cases, the multi-user behavior engine 434 can be implemented by a ML model, such as one or more neural networks. In some cases, the multi-user behavior engine 434 can send predictive information to behavior indication engine 410 that correspond to characteristics of the user and/or contexts frequently experience by the user. In some examples, the predictive information can be used to train the behavior indication engine 410 (e.g., when implemented by an ML model, such as a neural network). In addition or alternatively, the multi-user behavior engine 434 can send likelihood determination information to one or more of intervention engine 420, likelihood engine 422, and intervention effectiveness engine 426 that corresponds to characteristics of the user and/or contexts frequently experience by the user.

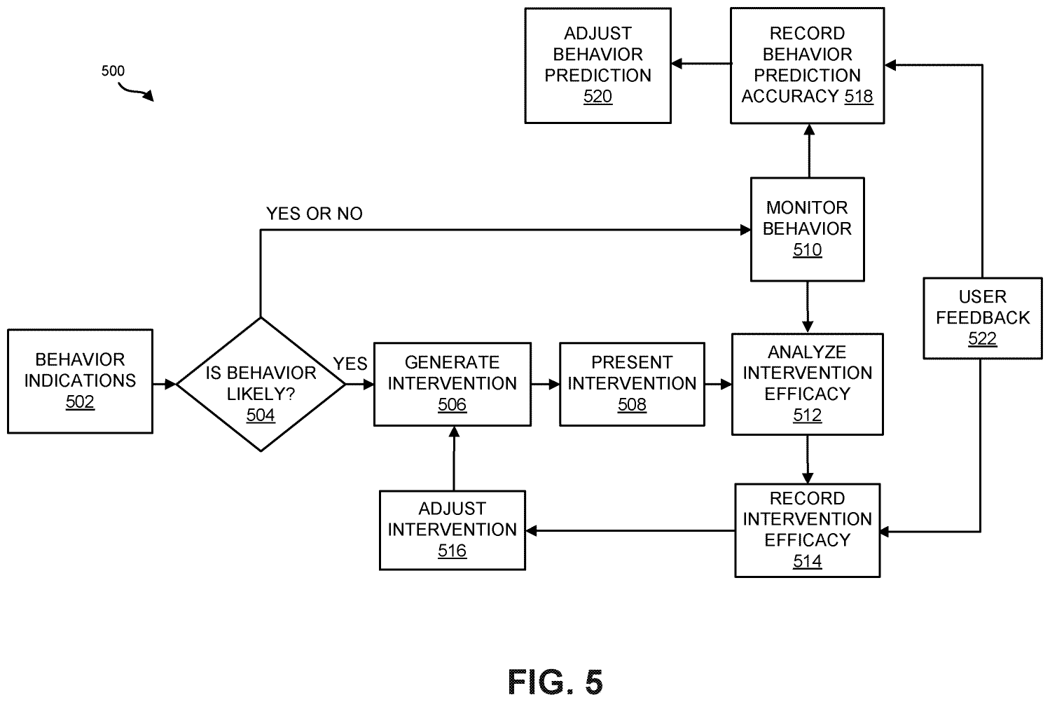

FIG. 5 is a flow diagram illustrating an example of a process 500 of generating one or more interventions. At block 502, the process 500 includes obtaining behavior indications associated with a user. In some cases, the behavior indications can include one or more of behavioral triggers, pre-behaviors, and behavioral artifacts (e.g., physical objects associated with the behavior). For example, the behavior indications can be obtained from a behavior indication engine system (e.g., behavior indication engine 410 shown in FIG. 4).

At block 504, the process 500 determines, based on the behavior indications received at block 502, whether an unwanted behavior is likely to occur or a desired behavior is unlikely to occur. For example, if the user has a goal to avoid eating sugary food, the process 500 can determine whether the behavior indications at a particular moment in time indicate that the user is likely to eat a sugary food. For instance, if the process 500 determines that the user is opening a freezer and reaching for ice cream, the process 500 may indicate that the likelihood of the user eating the ice cream is high. In some cases, the process 500 can determine whether the determined likelihood exceeds a predetermined threshold. Similarly, the process 500 can determine the likelihood that wanted behavior is unlikely to occur and determine whether the likelihood exceeds a predetermined threshold. In some implementations, process 500 can proceed to block 510 regardless of whether the behavior is determined to be likely at block 504. In some aspects, process 500 can perform block 510 in parallel with block 506 and/or block 508. In some aspects, the process 500 can proceed to block 506 if the process 500 determines that the likelihood of the behavior occurring (or not occurring) exceeds the threshold. In some cases, the process 500 can determine the likely effectiveness of an intervention associated with a behavior that is likely to occur or not occur (e.g., the likelihood of whether an intervention will prevent the unwanted behavior or promote the desired behavior in a given context or environment). For example, the process 500 can determine that an intervention that encourages a user to go for a walk will likely be effective if presented when the user stands up, as the likelihood of a user going for a walk when they stand up is greater than if the intervention is presented when the user is still sitting.

At block 506, the process generates an intervention to influence the user's behavior toward the user's stated goal. For example, in the case that process 500 determines that an unwanted behavior is likely to occur at block 504, the process 500 can generate an intervention that is likely to discourage the user from engaging in the unwanted behavior. In another example, in the case that process 500 at block 504 determines that the user is likely to avoid a desired behavior, the process 500 at block 506 can determine an intervention that is likely to encourage the user to engage in the behavior. In some cases, in addition or alternatively to generating the intervention based on the determined likelihood, the process 500 can generate the intervention based on previous success or failure of the available interventions in deterring (or encouraging) the user's target behavior. In some cases, the determined intervention can be selected from a library of intervention options for the user (e.g., obtained from intervention engine 424 shown in FIG. 4). In some cases, the intervention options can be ranked based on effectiveness of the interventions on previous occasions. In some cases, the determined intervention can be selected from a library of intervention options determined based on intervention efficacy determined from multiple users (e.g., obtained from multi-user intervention engine 432 shown in FIG. 4).

In some cases, the specific intervention generated by the process 500 can be based on the determined likelihood that the behavior will occur (or will not occur). For example, if the process 500 determines at block 504 that the likelihood for a particular behavior to occur (or not occur) exceeds the likelihood threshold by a small amount, the process 500 can determine that a minor intervention is likely to deter the user from (or encourage the user to) engage in the behavior. In one illustrative example, a minor intervention could include presenting a progress intervention reminding the user how long they have successfully abstained from the unwanted behavior (e.g., intervention 106 shown in FIG. 1B, intervention 112 shown in FIG. 1E, or the like). In some aspects, if the process 500 determines at block 504 that the likelihood for the behavior to occur (or not occur) exceeds the likelihood threshold by a large amount, the process 500 can determine that only a major intervention is likely to deter the user from engaging in the behavior (or encourage the user to engage in the behavior). In one illustrative example, a major intervention could include notifying a designated support person, playing an audio message, or the like. In some cases, the use of minor or major interventions may only be utilized for certain behaviors (e.g., a minor intervention may be useful in preventing the consumption of certain amount of food) and may not be utilized for other types of behaviors (e.g., a minor intervention may not deter a person with drinking problems from drinking alcohol).

In some cases, the process 500 can determine the intervention based on indications of success of interventions for individuals other than the user, but that share one or more characteristics with the user. In some cases, the process 500 can obtain one or more interventions from a multi-user intervention library (e.g., obtained from multi-user intervention engine 432 shown in FIG. 4) located on a server. In some cases, the multi-user intervention library can include indications of intervention effectiveness associated with users having common user characteristics (e.g., family status, personality type, any other characteristic described herein, and/or any other characteristic that can be common to different users). In some cases, in addition to or as an alternative to associating intervention effectiveness with user characteristics, the multi-user intervention library can include indications of intervention effectiveness associated with users commonly experiencing similar contexts. In some cases, the process 500 can obtain one or more interventions obtained from the multi-user intervention library located on the server that were previously effective for other individuals sharing similar characteristics with the user and/or other individuals frequently experiencing similar context as the user.

At block 508, the process 500 presents the intervention determined at block 508 to the user. The intervention presented to the user can include, but is not limited to, any of the interventions described in the present disclosure, such as interventions 106, 108, 110, 112, 124, 126, 128, and 142 shown in FIG. 1B through FIG. 1G, audio interventions, contacting a designated support person, or the like. In some cases, the process 500 can generate an intervention and send the intervention to another device to present the intervention to the user.

At block 510, the process 500 monitors whether the behavior for which the likelihood was determined at block 504 occurs or does not occur. For example, if the process 500 determines at block 504 that an unwanted behavior is likely, determines an intervention at block 506, and presents the intervention at block 508, the process 500 can determine at block 510 whether the unwanted behavior occurs after presenting the intervention. In another example, if the process 500 determines that an unwanted behavior is unlikely at block 504, the process 500 can monitor for the unwanted behavior to determine whether the unwanted behavior occurs.

At block 512, the process 500 analyzes the efficacy of the intervention presented to the user at block 508. For example, the process 500 at block 512 can determine whether the target behavior occurred (or did not occur) after presenting the intervention at block 508. In some cases, the process 500 can determine additional impacts of the intervention. In one illustrative example, the process 500 can determine whether a pre-behavior that was expected to occur based on the likelihood determination at 504 was prevented by the intervention presented at block 508. In another illustrative example, the process 500 can determine whether the intervention was at least partially successful. For example, if the user performs an unwanted behavior to a lesser degree than in previous instances (e.g., smoking one cigarette instead of two), the process 500 can determine that the intervention was partially effective.

At block 514, the process 500 can record the intervention efficacy analyzed at block 512. In some examples, user feedback 522 can be utilized by the process 500 at block 514. For instance, a user can provide input indicating how effective an intervention was in the user's opinion, indicating whether the user engaged in the behavior, and/or provide other feedback. In some cases, one or more additional parameters can be recorded along with the intervention efficacy to provide context to the recorded intervention efficacy. For example, one or more of the behavior indications evaluated at block 504 can be stored along with the recorded intervention efficacy to provide additional context to the stored intervention efficacy.

At block 516, the process 500 can adjust the interventions for the user based on the intervention efficacy determined at block 512. In one illustrative example, adjusting the intervention can include adjusting a score associated with the intervention. In some cases, a higher score can indicate a higher likelihood that the intervention will be successful in the future. In some cases, each intervention can be associated with multiple different scores, where each of the scores can be associated with a different context. For example, a particular intervention may be effective for a first context (e.g., before the user has decided to engage in the target behavior), but may be ineffective for a second context (e.g., once the user has started to engage in pre-behaviors and/or decided to engage in the behavior). In such an example, the particular intervention can have a high score for the first context and a low score for the second context. In some cases, the process 500 can adjust the intervention based on one or more of the specific context evaluated at block 504 and the intervention efficacy determined at block 512. In some cases, the process 500 can provide indications of intervention effectiveness to a system that determines the effectiveness of interventions for multiple different users (e.g., multi-user engine 430 shown in FIG. 4). In some cases, the process 500 can adjust the interventions for the user based on one or more interventions obtained from the multi-user intervention library located on the server that were previously effective for other users sharing similar characteristics with the user and/or other users frequently experiencing similar context as the user. In some cases, the intervention adjustment can include progressively adjusting the intervention (e.g., in real-time) based on effectiveness. For example, a system (e.g., an XR system) can present a first level of intervention that includes an image of a user's family on a pack of cigarettes. If the user reaches for and picks up the cigarettes, the system can then present a more intense intervention that includes a pre-recorded message from a family member.

At block 518, the process 500 can record behavior prediction accuracy. For example, if the process 500 determines that the behavior is unlikely to occur at block 504 and does not provide an intervention, but the process 500 detects that the behavior did occur at block 510, the process 500 at block 518 can record a behavior prediction failure. In some cases, the process 500 can record a behavior prediction success when the process 500 determines that the behavior is likely at block 504 and detects that the behavior did occur at block 510,. In some cases, one or more additional parameters can be recorded along with the behavior prediction to provide context to the recorded behavior prediction. For example, one or more of the behavior indications evaluated at block 504 can be stored along with the recorded behavior prediction to provide additional context to the stored behavior prediction. In some examples, the user feedback 522 can be utilized by the process 500 at block 518. For instance, a user can provide input indicating how accurate a behavior prediction was.

At block 520, the process 500 can adjust the behavior prediction (e.g., by adjusting a likelihood algorithm) for the user based on the behavior prediction accuracy recorded at block 518. In one illustrative example, adjusting the behavior prediction can include adjusting one or more weightings associated with the input behavior indications obtained at block 502. In some cases, the process 500 can indicate behavior prediction failure or success to a behavior indication engine (e.g., behavior indication engine 410 shown in FIG. 4) for use in refining the behavior triggers, pre-behaviors, and/or behavior artifacts for which the behavior indication engine is monitoring. In some cases, the process 500 can provide behavior indications to a server that determines the accuracy of behavior prediction and/or behavior information (e.g., behavioral triggers, pre-behaviors, and/or behavioral artifacts) for multiple different users (e.g., obtained from multi-user engine 430 shown in FIG. 4). In some cases, at block 520 the process 500 can adjust the behavior prediction for a user having certain characteristics and/or experiencing a particular context based on indications of accuracy of determining the likelihood of other users engaging in the same behavior or a similar behavior, where the other users share similar characteristics with the user and/or the other users previously experienced a similar context to the user. In some cases, the process 500 can continue to block 504 after the adjustment at block 520 is performed.

As noted above, the behavioral intervention management system 400 and related techniques described herein can allow a system to detect the likelihood of a behavior occurring or not occurring and provide an intervention to encourage or discourage a user to engage in the behavior depending on a user's goals. The behavioral intervention management system 400 can learn the behavioral indicators specific to a user that a target behavior is likely to occur (or not occur) in the future and the interventions specific to a user that are likely to alter the user's behavior in a desired way (e.g., encouraging the user to engage in a desired behavior, or discouraging the user from engaging in an undesired behavior). Providing interventions before a behavior occurs or does not occur can increase the chance that the user will choose to act in a way that is consistent with the user's goals.

The behavioral intervention management system 400 can also learn behavioral indicators and interventions for multiple users and determine behavior indicators and interventions that are applicable to and likely to be successful for subsets of the user population. For example, the behavioral intervention management system 400 can associate user characteristics and/or goals with likelihood of success of a particular intervention. Example characteristics include, but are not limited to, gender, age, family status, target behavior, severity of problem, country, culture, personality type, one or more health conditions, one or more dietary restrictions, one or more physical capabilities, and/or other characteristic. In addition to or as an alternative to associating user characteristics and/or goals with likelihood of success of a particular intervention, the behavioral intervention management system 400 can associate contexts (e.g., as represented by contextual information) with likelihood of success of a particular intervention. Example contextual information includes, but are not limited to, time of day, locale of the user (e.g., at work, at home, near a bar), the combination of technologies and/or sensors in use (e.g., whether the devices in the vicinity of the user are capable of providing an indicated intervention), stress level, other people near the person, or the like. In some cases, the behavioral intervention management system 400 can similarly associate characteristics and/or contexts with behavioral information and the success or failure of behavior likelihood predictions (e.g., at block 504 of process 500 shown in FIG. 5) to learn predictive behaviors for subsets of the user population.

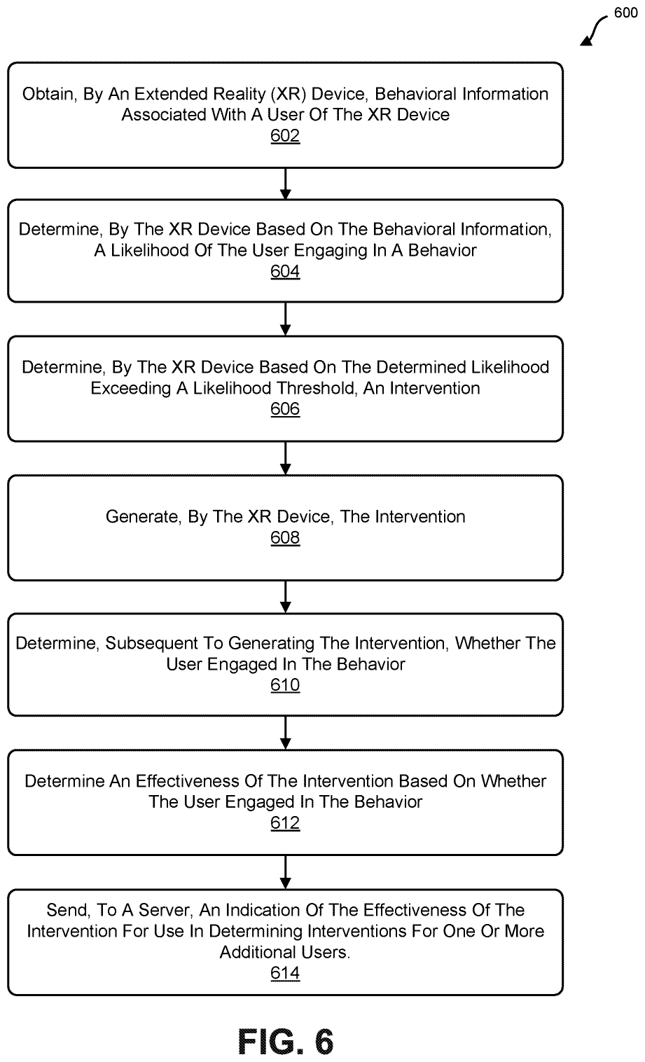

FIG. 6 is a flow diagram illustrating a process 600 for generating one or more interventions. At block 602, the process 600 includes obtaining, by an extended reality (XR) device, behavioral information associated with a user of the XR device. For instance, to obtain the behavioral information associated with the user of the XR device, the process 600 can determine one or more behavioral triggers that are predictive of the behavior. The one or more behavioral triggers can include a stress level of the user, a heart rate of the user, an object within a field of view of the XR device, a location and/or environment (e.g., objects, people, etc. in the environment) at which the user is located, a time at which the behavioral information is obtained, one or more people in proximity to the user, an activity in which the user is engaged, any combination thereof, and/or other triggers. In some cases, to obtain the behavioral information associated with the user of the XR device, the process 600 can determine one or more pre-behaviors indicative of a likelihood of the user engaging in the behavior, as described herein. In some examples, to obtain the behavioral information associated with the user of the XR device, the process 600 can include detecting, in one or more images obtained by the XR device, one or more behavioral artifacts associated with the behavior.

At block 604, the process 600 includes determining, by the XR device based on the behavioral information, a likelihood of the user engaging in a behavior. In some cases, the process 500 can determine the likely effectiveness of an intervention associated with a behavior that is likely to occur or not occur (e.g., the likelihood of whether an intervention will prevent the unwanted behavior or promote the desired behavior in a given context or environment). At block 606, the process 600 includes determining, by the XR device based on the determined likelihood exceeding a likelihood threshold, an intervention associated with the behavior. In some examples, the process 600 can include determining, by the XR device based on the determined likelihood falling below the likelihood threshold, to forego generating a particular intervention. In some cases, the process 600 at block 606 can determine the intervention or determine to forego generating a particular intervention based on the likely effectiveness of the intervention. For example, the process 500 can determine that an intervention that encourages a user to go for a walk will likely be effective if presented when the user is standing up, but may determine that the intervention will not be effective if the user is sifting or lying down.

At block 608, the process 600 includes generating, by the XR device, the intervention. For instance, to generate the intervention, the process 600 can display virtual content on a display of the XR device. A real-world environment is viewable through the display of the XR device as the virtual content is displayed by the display. In another example, the process 600 can output audio (e.g., using a speaker) associated with the intervention. Generating the intervention can include any other type of output.

At block 610, the process 600 includes determining, subsequent to outputting the intervention, whether the user engaged in the behavior. Whether the user engaged in the behavior can be based on analysis of one or more images captured by the XR device, user input, and/or other information. At block 612, the process 600 includes determining an effectiveness of the intervention based on whether the user engaged in the behavior. For example, the process 600 can determine an intervention is effective if the intervention prevented the user from performing an unwanted behavior (e.g., eating a bag of chips) or resulted in the user performing a wanted behavior (e.g., exercise).

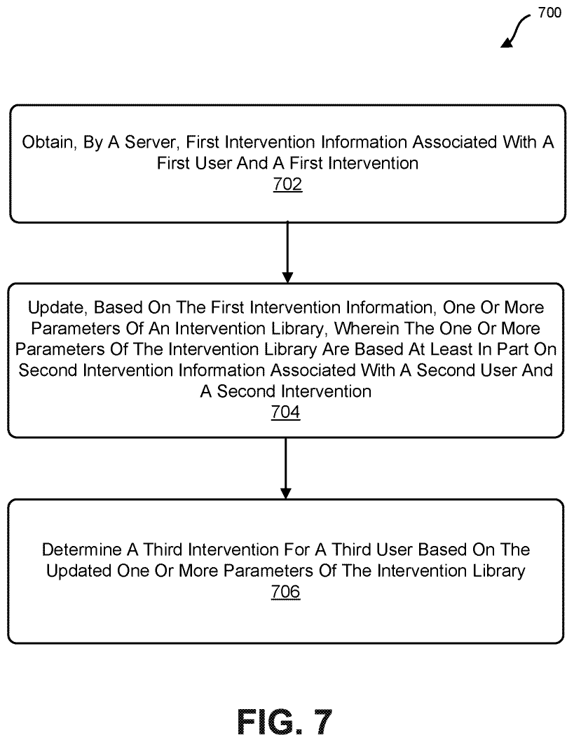

At block 614, the process 600 includes sending, to a server, an indication of the effectiveness of the intervention for use in determining interventions for one or more additional users. In some cases, the process 600 includes sending, to the server, an indication of an accuracy of determining the likelihood of the user engaging in the behavior (e.g., for use in determining likelihoods of engaging in the behavior for one or more additional users). In some examples, the process 600 includes sending, to the server, contextual information associated with the intervention. For example, as described herein, the contextual information associated with the intervention can include a time of day, one or more actions by the user of the XR device prior to the intervention, the behavioral information associated with the user of the XR device, a location of the user of the XR device, a proximity of the user of the XR device to one or more individuals, any combination thereof, and/or other information. In some examples, the process 600 includes sending, to the server, one or more characteristics associated with the user of the XR device. For instance, as described herein, the one or more characteristics associated with the user of the XR device can include gender, age, family status, target behavior, country, culture, locale, personality type, one or more health conditions, one or more dietary restrictions, one or more physical capabilities, any combination thereof, and/or other characteristics. Example operations of a server are described herein, including below with respect to FIG. 7.

FIG. 7 is a flow diagram illustrating a process 700 for predicting one or more behaviors. At block 702, the process 700 includes obtaining, by a server, first intervention information associated with a first user and a first intervention. In some examples, the first intervention information associated with the first user includes an intervention type, an indication of an effectiveness of the first intervention, an intervention context associated with the first intervention, one or more characteristics associated with the first user, any combination thereof, and/or other information. In some cases, the first intervention information includes contextual information associated with the first intervention. For instance, the contextual information associated with the first intervention can include a time of day, one or more actions by the first user prior to the first intervention, the first intervention information associated with the first user, a location the first user, a proximity of the first user to one or more individuals, any combination thereof, and/or other contextual information. In some aspects, the first intervention information includes one or more characteristics associated with the first user. For instance, the one or more characteristics associated with the first user can include gender, age, family status, target behavior, country, culture, locale, personality type, any combination thereof, and/or other characteristics.

At block 704, the process 700 includes updating, based on the first intervention information, one or more parameters of an intervention library. The one or more parameters of the intervention library are based at least in part on second intervention information associated with a second user and a second intervention. For example, the parameter(s) of the intervention library can be updated based on multiple interventions for multiple users. The intervention library can then be used to determine or recommend interventions for additional users. For instance, at block 706, the process 700 includes determining a third intervention for a third user based on the updated one or more parameters of the intervention library.

In some examples, to determine the third intervention for the third user based on the updated one or more parameters of the intervention library, the process 700 includes obtaining, by the server, third intervention information associated with the third user. The process 700 can include determining a correlation between the third intervention information associated with the third user and fourth intervention information associated with the intervention library. The process 700 can further include determining, based on the correlation between the third intervention information and the fourth intervention information exceeding a correlation threshold, the third intervention. The process 700 can include sending, to a device associated with the third user, the third intervention. The device associated with the third user can then present (e.g., display, output via a speaker, etc.) the third intervention to the third user.

In some aspects, the process 700 includes obtaining, by the server, fifth behavioral information associated with a fifth user and a fifth behavior. The process 700 can include updating, based on the fifth behavioral information, one or more parameters of a behavior library. The one or more parameters of the behavior library may be based at least in part on sixth behavioral information associated with a sixth user. The process 700 can further include determining one or more behavior parameters for a sixth user based on the updated one or more parameters of the intervention library. In some cases, the one or more behavior parameters for the sixth user include behavioral triggers, pre-behaviors, behavioral artifacts associated with the fifth behavior, any combination thereof, and/or other parameters. In some aspects, the one or more behavior parameters for the sixth user include one or more weightings associated with determining a likelihood that the sixth user will perform or not perform the fifth behavior. In some examples, the fifth behavioral information includes one or more characteristics associated with the fifth user. In some cases, the fifth behavioral information includes contextual information associated with the fifth behavior.

In some examples, the processes described herein (e.g., processes 500, 600, 700 and/or other process described herein) may be performed by a computing device or apparatus. In one example, one or more of the processes can be performed by the XR system 200 shown in FIG. 2. In another example, one or more of the processes can be performed by the XR system 320 shown in FIG. 3. In another example, one or more of the processes can be performed by the computing system 1000 shown in FIG. 10. For instance, a computing device with the computing system 1000 shown in FIG. 10 can include the components of the XR system 200 and can implement the operations of the process 500 of FIG. 5, the process 600 of FIG. 6, the process 700 of FIG. 7, and/or other processes described herein.

The computing device can include any suitable device, such as a vehicle or a computing device of a vehicle, a mobile device (e.g., a mobile phone), a desktop computing device, a tablet computing device, a wearable device (e.g., a VR headset, an AR headset, AR glasses, a network-connected watch or smartwatch, or other wearable device), a server computer, a robotic device, a television, and/or any other computing device with the resource capabilities to perform the processes described herein, including the processes 500, 600, 700, and/or other process described herein. In some cases, the computing device or apparatus may include various components, such as one or more input devices, one or more output devices, one or more processors, one or more microprocessors, one or more microcomputers, one or more cameras, one or more sensors, and/or other component(s) that are configured to carry out the steps of processes described herein. In some examples, the computing device may include a display, a network interface configured to communicate and/or receive the data, any combination thereof, and/or other component(s). The network interface may be configured to communicate and/or receive Internet Protocol (IP) based data or other type of data.

The components of the computing device can be implemented in circuitry. For example, the components can include and/or can be implemented using electronic circuits or other electronic hardware, which can include one or more programmable electronic circuits (e.g., microprocessors, graphics processing units (GPUs), digital signal processors (DSPs), central processing units (CPUs), and/or other suitable electronic circuits), and/or can include and/or be implemented using computer software, firmware, or any combination thereof, to perform the various operations described herein.

The processes 500, 600, and 700 are illustrated as logical flow diagrams, the operation of which represents a sequence of operations that can be implemented in hardware, computer instructions, or a combination thereof. In the context of computer instructions, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, the processes 500, 600, and 700 and/or other process described herein may be performed under the control of one or more computer systems configured with executable instructions and may be implemented as code (e.g., executable instructions, one or more computer programs, or one or more applications) executing collectively on one or more processors, by hardware, or combinations thereof. As noted above, the code may be stored on a computer-readable or machine-readable storage medium, for example, in the form of a computer program comprising a plurality of instructions executable by one or more processors. The computer-readable or machine-readable storage medium may be non-transitory.

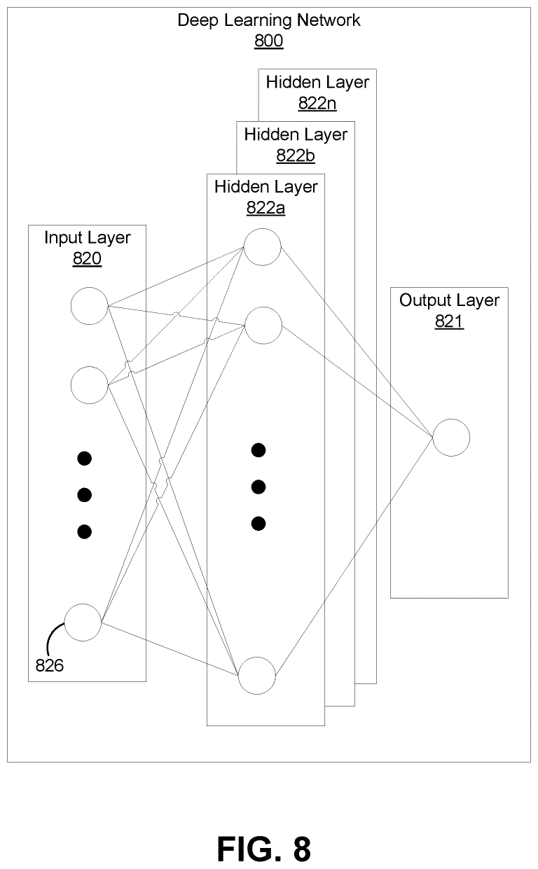

As noted above, various aspects of the present disclosure can use machine learning models or systems. FIG. 8 is an illustrative example of a deep learning neural network 800 that can be used to implement the machine learning based feature extraction and/or activity recognition (or classification) described above. An input layer 820 includes input data. In one illustrative example, the input layer 820 can include data representing the pixels of an input video frame. The neural network 800 includes multiple hidden layers 822a, 822b, through 822n. The hidden layers 822a, 822b, through 822n include “n” number of hidden layers, where “n” is an integer greater than or equal to one. The number of hidden layers can be made to include as many layers as needed for the given application. The neural network 800 further includes an output layer 821 that provides an output resulting from the processing performed by the hidden layers 822a, 822b, through 822n. In one illustrative example, the output layer 821 can provide a classification for an object in an input video frame. The classification can include a class identifying the type of activity (e.g., looking up, looking down, closing eyes, yawning, etc.).

The neural network 800 is a multi-layer neural network of interconnected nodes. Each node can represent a piece of information. Information associated with the nodes is shared among the different layers and each layer retains information as information is processed. In some cases, the neural network 800 can include a feed-forward network, in which case there are no feedback connections where outputs of the network are fed back into itself. In some cases, the neural network 800 can include a recurrent neural network, which can have loops that allow information to be carried across nodes while reading in input.

Information can be exchanged between nodes through node-to-node interconnections between the various layers. Nodes of the input layer 820 can activate a set of nodes in the first hidden layer 822a. For example, as shown, each of the input nodes of the input layer 820 is connected to each of the nodes of the first hidden layer 822a. The nodes of the first hidden layer 822a can transform the information of each input node by applying activation functions to the input node information. The information derived from the transformation can then be passed to and can activate the nodes of the next hidden layer 822b, which can perform their own designated functions. Example functions include convolutional, up-sampling, data transformation, and/or any other suitable functions. The output of the hidden layer 822b can then activate nodes of the next hidden layer, and so on. The output of the last hidden layer 822n can activate one or more nodes of the output layer 821, at which an output is provided. In some cases, while nodes (e.g., node 826) in the neural network 800 are shown as having multiple output lines, a node has a single output and all lines shown as being output from a node represent the same output value.

In some cases, each node or interconnection between nodes can have a weight that is a set of parameters derived from the training of the neural network 800. Once the neural network 800 is trained, it can be referred to as a trained neural network, which can be used to classify one or more activities. For example, an interconnection between nodes can represent a piece of information learned about the interconnected nodes. The interconnection can have a tunable numeric weight that can be tuned (e.g., based on a training dataset), allowing the neural network 800 to be adaptive to inputs and able to learn as more and more data is processed.

The neural network 800 is pre-trained to process the features from the data in the input layer 820 using the different hidden layers 822a, 822b, through 822n in order to provide the output through the output layer 821. In an example in which the neural network 800 is used to identify activities being performed by a driver in frames, the neural network 800 can be trained using training data that includes both frames and labels, as described above. For instance, training frames can be input into the network, with each training frame having a label indicating the features in the frames (for the feature extraction machine learning system) or a label indicating classes of an activity in each frame. In one example using object classification for illustrative purposes, a training frame can include an image of a number 2, in which case the label for the image can be [0 0 1 0 0 0 0 0 0 0].

In some cases, the neural network 800 can adjust the weights of the nodes using a training process called backpropagation. As noted above, a backpropagation process can include a forward pass, a loss function, a backward pass, and a weight update. The forward pass, loss function, backward pass, and parameter update is performed for one training iteration. The process can be repeated for a certain number of iterations for each set of training images until the neural network 800 is trained well enough so that the weights of the layers are accurately tuned.

For the example of identifying objects in frames, the forward pass can include passing a training frame through the neural network 800. The weights are initially randomized before the neural network 800 is trained. As an illustrative example, a frame can include an array of numbers representing the pixels of the image. Each number in the array can include a value from 0 to 255 describing the pixel intensity at that position in the array. In one example, the array can include a 28×28×3 array of numbers with 28 rows and 28 columns of pixels and 3 color components (such as red, green, and blue, or luma and two chroma components, or the like).

As noted above, for a first training iteration for the neural network 800, the output will likely include values that do not give preference to any particular class due to the weights being randomly selected at initialization. For example, if the output is a vector with probabilities that the object includes different classes, the probability value for each of the different classes may be equal or at least very similar (e.g., for ten possible classes, each class may have a probability value of 0.1). With the initial weights, the neural network 800 is unable to determine low level features and thus cannot make an accurate determination of what the classification of the object might be. A loss function can be used to analyze error in the output. Any suitable loss function definition can be used, such as a Cross-Entropy loss. Another example of a loss function includes the mean squared error (MSE), defined as Eₜₒₜₐₗ=Σ1/2(target−output)². The loss can be set to be equal to the value of Eₜₒₜₐₗ.

The loss (or error) will be high for the first training images since the actual values will be much different than the predicted output. The goal of training is to minimize the amount of loss so that the predicted output is the same as the training label. The neural network 800 can perform a backward pass by determining which inputs (weights) most contributed to the loss of the network, and can adjust the weights so that the loss decreases and is eventually minimized. A derivative of the loss with respect to the weights (denoted as dL/dW, where W are the weights at a particular layer) can be computed to determine the weights that contributed most to the loss of the network. After the derivative is computed, a weight update can be performed by updating all the weights of the filters. For example, the weights can be updated so that they change in the opposite direction of the gradient. The weight update can be denoted as w=wᵢ−ηdL/dW, where w denotes a weight, wᵢ denotes the initial weight, and η denotes a learning rate. The learning rate can be set to any suitable value, with a high learning rate including larger weight updates and a lower value indicating smaller weight updates.

The neural network 800 can include any suitable deep network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. The neural network 800 can include any other deep network other than a CNN, such as an autoencoder, a deep belief nets (DBNs), a Recurrent Neural Networks (RNNs), among others.

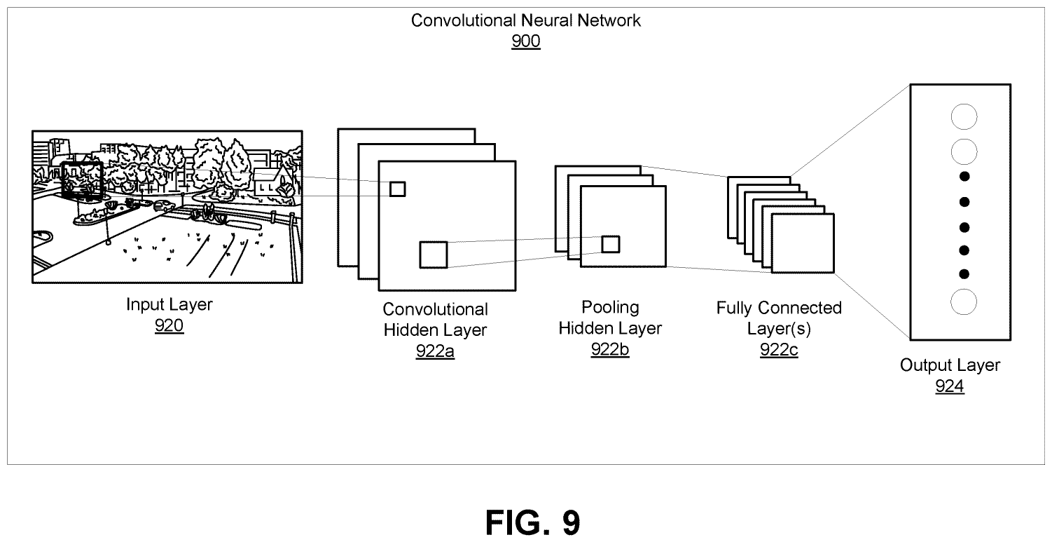

FIG. 9 is an illustrative example of a convolutional neural network (CNN) 900. The

input layer 920 of the CNN 900 includes data representing an image or frame. For example, the data can include an array of numbers representing the pixels of the image, with each number in the array including a value from 0 to 255 describing the pixel intensity at that position in the array. Using the previous example from above, the array can include a 28×28×3 array of numbers with 28 rows and 28 columns of pixels and 3 color components (e.g., red, green, and blue, or luma and two chroma components, or the like). The image can be passed through a convolutional hidden layer 922a, an optional non-linear activation layer, a pooling hidden layer 922b, and fully connected hidden layers 922c to get an output at the output layer 924. While only one of each hidden layer is shown in FIG. 9, one of ordinary skill will appreciate that multiple convolutional hidden layers, non-linear layers, pooling hidden layers, and/or fully connected layers can be included in the CNN 900. As previously described, the output can indicate a single class of an object or can include a probability of classes that best describe the object in the image.

The first layer of the CNN 900 is the convolutional hidden layer 922a. The convolutional hidden layer 922a analyzes the image data of the input layer 920. Each node of the convolutional hidden layer 922a is connected to a region of nodes (pixels) of the input image called a receptive field. The convolutional hidden layer 922a can be considered as one or more filters (each filter corresponding to a different activation or feature map), with each convolutional iteration of a filter being a node or neuron of the convolutional hidden layer 922a. For example, the region of the input image that a filter covers at each convolutional iteration would be the receptive field for the filter. In one illustrative example, if the input image includes a 28×28 array, and each filter (and corresponding receptive field) is a 5×5 array, then there will be 24×24 nodes in the convolutional hidden layer 922a. Each connection between a node and a receptive field for that node learns a weight and, in some cases, an overall bias such that each node learns to analyze its particular local receptive field in the input image. Each node of the hidden layer 922a will have the same weights and bias (called a shared weight and a shared bias). For example, the filter has an array of weights (numbers) and the same depth as the input. A filter will have a depth of 3 for the video frame example (according to three color components of the input image). An illustrative example size of the filter array is 5×5×3, corresponding to a size of the receptive field of a node.

The convolutional nature of the convolutional hidden layer 922a is due to each node of the convolutional layer being applied to its corresponding receptive field. For example, a filter of the convolutional hidden layer 922a can begin in the top-left corner of the input image array and can convolve around the input image. As noted above, each convolutional iteration of the filter can be considered a node or neuron of the convolutional hidden layer 922a. At each convolutional iteration, the values of the filter are multiplied with a corresponding number of the original pixel values of the image (e.g., the 5×5 filter array is multiplied by a 5×5 array of input pixel values at the top-left corner of the input image array). The multiplications from each convolutional iteration can be summed together to obtain a total sum for that iteration or node. The process is next continued at a next location in the input image according to the receptive field of a next node in the convolutional hidden layer 922a. For example, a filter can be moved by a step amount (referred to as a stride) to the next receptive field. The stride can be set to 1 or other suitable amount. For example, if the stride is set to 1, the filter will be moved to the right by 1 pixel at each convolutional iteration. Processing the filter at each unique location of the input volume produces a number representing the filter results for that location, resulting in a total sum value being determined for each node of the convolutional hidden layer 922a.

The mapping from the input layer to the convolutional hidden layer 922a is referred to as an activation map (or feature map). The activation map includes a value for each node representing the filter results at each locations of the input volume. The activation map can include an array that includes the various total sum values resulting from each iteration of the filter on the input volume. For example, the activation map will include a 24×24 array if a 5×5 filter is applied to each pixel (a stride of 1) of a 28×28 input image. The convolutional hidden layer 922a can include several activation maps in order to identify multiple features in an image. The example shown in FIG. 9 includes three activation maps. Using three activation maps, the convolutional hidden layer 922a can detect three different kinds of features, with each feature being detectable across the entire image.

In some examples, a non-linear hidden layer can be applied after the convolutional hidden layer 922a. The non-linear layer can be used to introduce non-linearity to a system that has been computing linear operations. One illustrative example of a non-linear layer is a rectified linear unit (ReLU) layer. A ReLU layer can apply the function f(x)=max(0, x) to all of the values in the input volume, which changes all the negative activations to 0. The ReLU can thus increase the non-linear properties of the CNN 900 without affecting the receptive fields of the convolutional hidden layer 922a.

The pooling hidden layer 922b can be applied after the convolutional hidden layer 922a (and after the non-linear hidden layer when used). The pooling hidden layer 922b is used to simplify the information in the output from the convolutional hidden layer 922a. For example, the pooling hidden layer 922b can take each activation map output from the convolutional hidden layer 922a and generates a condensed activation map (or feature map) using a pooling function. Max-pooling is one example of a function performed by a pooling hidden layer. Other forms of pooling functions be used by the pooling hidden layer 922a, such as average pooling, L2-norm pooling, or other suitable pooling functions. A pooling function (e.g., a max-pooling filter, an L2-norm filter, or other suitable pooling filter) is applied to each activation map included in the convolutional hidden layer 922a. In the example shown in FIG. 9, three pooling filters are used for the three activation maps in the convolutional hidden layer 922a.

In some examples, max-pooling can be used by applying a max-pooling filter (e.g.,

having a size of 2×2) with a stride (e.g., equal to a dimension of the filter, such as a stride of 2) to an activation map output from the convolutional hidden layer 922a. The output from a max-pooling filter includes the maximum number in every sub-region that the filter convolves around. Using a 2×2 filter as an example, each unit in the pooling layer can summarize a region of 2×2 nodes in the previous layer (with each node being a value in the activation map). For example, four values (nodes) in an activation map will be analyzed by a 2×2 max-pooling filter at each iteration of the filter, with the maximum value from the four values being output as the “max” value. If such a max-pooling filter is applied to an activation filter from the convolutional hidden layer 922a having a dimension of 24×24 nodes, the output from the pooling hidden layer 922b will be an array of 12×12 nodes.

In some examples, an L2-norm pooling filter could also be used. The L2-norm pooling filter includes computing the square root of the sum of the squares of the values in the 2×2 region (or other suitable region) of an activation map (instead of computing the maximum values as is done in max-pooling), and using the computed values as an output.

Intuitively, the pooling function (e.g., max-pooling, L2-norm pooling, or other pooling function) determines whether a given feature is found anywhere in a region of the image, and discards the exact positional information. This can be done without affecting results of the feature detection because, once a feature has been found, the exact location of the feature is not as important as its approximate location relative to other features. Max-pooling (as well as other pooling methods) offer the benefit that there are many fewer pooled features, thus reducing the number of parameters needed in later layers of the CNN 900.

The final layer of connections in the network is a fully-connected layer that connects

every node from the pooling hidden layer 922b to every one of the output nodes in the output layer 924. Using the example above, the input layer includes 28×28 nodes encoding the pixel intensities of the input image, the convolutional hidden layer 922a includes 3×24×24 hidden feature nodes based on application of a 5×5 local receptive field (for the filters) to three activation maps, and the pooling hidden layer 922b includes a layer of 3×12×12 hidden feature nodes based on application of max-pooling filter to 2×2 regions across each of the three feature maps. Extending this example, the output layer 924 can include ten output nodes. In such an example, every node of the 3×12×12 pooling hidden layer 922b is connected to every node of the output layer 924.

The fully connected layer 922c can obtain the output of the previous pooling hidden layer 922b (which should represent the activation maps of high-level features) and determines the features that most correlate to a particular class. For example, the fully connected layer 922c layer can determine the high-level features that most strongly correlate to a particular class, and can include weights (nodes) for the high-level features. A product can be computed between the weights of the fully connected layer 922c and the pooling hidden layer 922b to obtain probabilities for the different classes. For example, if the CNN 900 is being used to predict that an object in a video frame is a person, high values will be present in the activation maps that represent high-level features of people (e.g., two legs are present, a face is present at the top of the object, two eyes are present at the top left and top right of the face, a nose is present in the middle of the face, a mouth is present at the bottom of the face, and/or other features common for a person).

In some examples, the output from the output layer 924 can include an M-dimensional vector (in the prior example, M=10). M indicates the number of classes that the CNN 900 has to choose from when classifying the object in the image. Other example outputs can also be provided. Each number in the M-dimensional vector can represent the probability the object is of a certain class. In one illustrative example, if a 10-dimensional output vector represents ten different classes of objects is [0 0 0.05 0.8 0 0.15 0 0 0 0], the vector indicates that there is a 5% probability that the image is the third class of object (e.g., a dog), an 80% probability that the image is the fourth class of object (e.g., a human), and a 15% probability that the image is the sixth class of object (e.g., a kangaroo). The probability for a class can be considered a confidence level that the object is part of that class.

FIG. 10 is a diagram illustrating an example of a system for implementing certain aspects of the present technology. In particular, FIG. 10 illustrates an example of computing system 1000, which can be for example any computing device making up internal computing system, a remote computing system, a camera, or any component thereof in which the components of the system are in communication with each other using connection 1005. Connection 1005 can be a physical connection using a bus, or a direct connection into processor 1010, such as in a chipset architecture. Connection 1005 can also be a virtual connection, networked connection, or logical connection.

In some embodiments, computing system 1000 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example computing system 1000 includes at least one processing unit (CPU or processor) 1010 and connection 1005 that couples various system components including system memory 1015, such as read-only memory (ROM) 1020 and random access memory (RAM) 1025 to processor 1010. Computing system 1000 can include a cache 1012 of high-speed memory connected directly with, in close proximity to, or integrated as part of processor 1010.

Processor 1010 can include any general purpose processor and a hardware service or software service, such as services 1032, 1034, and 1036 stored in storage device 1030, configured to control processor 1010 as well as a special-purpose processor where software instructions are incorporated into the actual processor design. Processor 1010 may essentially be a completely self-contained computing system, containing multiple cores or processors, a bus, memory controller, cache, etc. A multi-core processor may be symmetric or asymmetric.

To enable user interaction, computing system 1000 includes an input device 1045, which can represent any number of input mechanisms, such as a microphone for speech, a touch-sensitive screen for gesture or graphical input, keyboard, mouse, motion input, speech, etc. Computing system 1000 can also include output device 1035, which can be one or more of a number of output mechanisms. In some instances, multimodal systems can enable a user to provide multiple types of input/output to communicate with computing system 1000. Computing system 1000 can include communications interface 1040, which can generally govern and manage the user input and system output. The communication interface may perform or facilitate receipt and/or transmission wired or wireless communications using wired and/or wireless transceivers, including those making use of an audio jack/plug, a microphone jack/plug, a universal serial bus (USB) port/plug, an Apple® Lightning® port/plug, an Ethernet port/plug, a fiber optic port/plug, a proprietary wired port/plug, a BLUETOOTH® wireless signal transfer, a BLUETOOTH® low energy (BLE) wireless signal transfer, an IBEACON® wireless signal transfer, a radio-frequency identification (RFID) wireless signal transfer, near-field communications (NFC) wireless signal transfer, dedicated short range communication (DSRC) wireless signal transfer, 802.11 Wi-Fi wireless signal transfer, wireless local area network (WLAN) signal transfer, Visible Light Communication (VLC), Worldwide Interoperability for

Microwave Access (WiMAX), Infrared (IR) communication wireless signal transfer, Public Switched Telephone Network (PSTN) signal transfer, Integrated Services Digital Network (ISDN) signal transfer, 3G/4G/5G/LTE cellular data network wireless signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof. The communications interface 1040 may also include one or more Global Navigation Satellite System (GNSS) receivers or transceivers that are used to determine a location of the computing system 1000 based on receipt of one or more signals from one or more satellites associated with one or more GNSS systems. GNSS systems include, but are not limited to, the US-based Global Positioning System (GPS), the Russia-based Global Navigation Satellite System (GLONASS), the China-based BeiDou Navigation Satellite System (BDS), and the Europe-based Galileo GNSS. There is no restriction on operating on any particular hardware arrangement, and therefore the basic features here may easily be substituted for improved hardware or firmware arrangements as they are developed.

Storage device 1030 can be a non-volatile and/or non-transitory and/or computer-readable memory device and can be a hard disk or other types of computer readable media which can store data that are accessible by a computer, such as magnetic cassettes, flash memory cards, solid state memory devices, digital versatile disks, cartridges, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity module (SIM) card, a mini/micro/nano/pico SIM card, another integrated circuit (IC) chip/card, random access memory (RAM), static RAM (SRAM), dynamic RAM (DRAM), read-only memory (ROM), programmable read-only memory (PROM), erasable programmable read-only memory (EPROM), electrically erasable programmable read-only memory (EEPROM), flash EPROM (FLASHEPROM), cache memory (L1/L2/L3/L4/L5/L#), resistive random-access memory (RRAM/ReRAM), phase change memory (PCM), spin transfer torque RAM (STT-RAM), another memory chip or cartridge, and/or a combination thereof.

The storage device 1030 can include software services, servers, services, etc., that when the code that defines such software is executed by the processor 1010, it causes the system to perform a function. In some embodiments, a hardware service that performs a particular function can include the software component stored in a computer-readable medium in connection with the necessary hardware components, such as processor 1010, connection 1005, output device 1035, etc., to carry out the function.

As used herein, the term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted using any suitable means including memory sharing, message passing, token passing, network transmission, or the like.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Specific details are provided in the description above to provide a thorough understanding of the embodiments and examples provided herein. However, it will be understood by one of ordinary skill in the art that the embodiments may be practiced without these specific details. For clarity of explanation, in some instances the present technology may be presented as including individual functional blocks including functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual embodiments may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc. Examples of computer-readable media that may be used to store instructions, information used, and/or information created during methods according to described examples include magnetic or optical disks, flash memory, USB devices provided with non-volatile memory, networked storage devices, and so on.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for

executing them, and other structures for supporting such computing resources are example means for providing the functions described in the disclosure.

In the foregoing description, aspects of the application are described with reference to specific embodiments thereof, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

The various illustrative logical blocks, modules, circuits, and algorithm steps described in connection with the embodiments disclosed herein may be implemented as electronic hardware, computer software, firmware, or combinations thereof To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans May implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor May be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

Illustrative aspects of the disclosure include:

Aspect 1: A method of generating one or more interventions, the method comprising: obtaining, by an extended reality (XR) device, behavioral information associated with a user of the XR device; determining, by the XR device based on the behavioral information, a likelihood of the user engaging in a behavior; determining, by the XR device based on the determined likelihood exceeding a likelihood threshold, an intervention; generating, by the XR device, the intervention; determining, subsequent to generating the intervention, whether the user engaged in the behavior; determining an effectiveness of the intervention based on whether the user engaged in the behavior; and sending, to a server, an indication of the effectiveness of the intervention for use in determining interventions for one or more additional users.

Aspect 2: The method of Aspect 1, further comprising: sending, to the server, contextual information associated with the intervention, wherein the contextual information associated with the intervention comprises at least one of a time of day, one or more actions by the user of the XR device prior to the intervention, the behavioral information associated with the user of the XR device, a location of the user of the XR device, and a proximity of the user of the XR device to one or more individuals.

Aspect 3: The method of any of Aspects 1 to 2, further comprising: sending, to the server, one or more characteristics associated with the user of the XR device, wherein the one or more characteristics associated with the user of the XR device comprise at least one of gender, age, family status, target behavior, country, culture, locale, personality type, one or more health conditions, one or more dietary restrictions, and one or more physical capabilities.

Aspect 4: The method of any of Aspects 1 to 3, wherein obtaining the behavioral information associated with the user of the XR device includes determining one or more behavioral triggers that are predictive of the behavior, wherein the one or more behavioral triggers include at least one of a stress level of the user, a heart rate of the user, an object within a field of view of the XR device, a location at which the user is located, a time at which the behavioral information is obtained, one or more people in proximity to the user, and an activity in which the user is engaged.

Aspect 5: The method of any of Aspects 1 to 4, wherein obtaining the behavioral

information associated with the user of the XR device includes determining one or more pre-behaviors indicative of a likelihood of the user engaging in the behavior.

Aspect 6: The method of any of Aspects 1 to 5, wherein obtaining the behavioral information associated with the user of the XR device includes detecting, in one or more images obtained by the XR device, one or more behavioral artifacts associated with the behavior.

Aspect 7: The method of any of Aspects 1 to 6, wherein generating the intervention comprises displaying virtual content on a display of the XR device, wherein a real-world environment is viewable through the display of the XR device as the virtual content is displayed by the display.

Aspect 8: An apparatus for generating one or more interventions, comprising: at least one memory; and at least one processor coupled to the at least one memory, the at least one processor configured to: obtain behavioral information associated with a user of the apparatus; determine, based on the behavioral information, a likelihood of the user engaging in a behavior; determine, based on the determined likelihood exceeding a likelihood threshold, an intervention associated with the behavior; generate the intervention; determine, subsequent to outputting the intervention, whether the user engaged in the behavior; determine an effectiveness of the intervention based on whether the user engaged in the behavior; and send, to a server, an indication of the effectiveness of the intervention for use in determining interventions for one or more additional users.

Aspect 9: The apparatus of Aspect 8, wherein the at least one processor is configured to: send, to the server, contextual information associated with the intervention, wherein the contextual information associated with the intervention comprises at least one of a time of day, one or more actions by the user of the apparatus prior to the intervention, the behavioral information associated with the user of the apparatus, a location of the user of the apparatus, and a proximity of the user of the apparatus to one or more individuals.

Aspect 10: The apparatus of any of Aspects 8 to 9, wherein the at least one processor is configured to: send, to the server, one or more characteristics associated with the user of the apparatus, wherein the one or more characteristics associated with the user of the apparatus comprise at least one of gender, age, family status, target behavior, country, culture, locale, personality type, one or more health conditions, one or more dietary restrictions, and one or more physical capabilities.

Aspect 11: The apparatus of any of Aspects 8 to 10, wherein, to obtain the behavioral information associated with the user of the apparatus, the at least one processor is configured to determine one or more behavioral triggers that are predictive of the behavior, wherein the one or more behavioral triggers include at least one of a stress level of the user, a heart rate of the user, an object within a field of view of the apparatus, a location at which the user is located, a time at which the behavioral information is obtained, one or more people in proximity to the user, and an activity in which the user is engaged.

Aspect 12: The apparatus of any of Aspects 8 to 11, wherein, to obtain the behavioral information associated with the user of the apparatus, the at least one processor is configured to determine one or more pre-behaviors indicative of a likelihood of the user engaging in the behavior.

Aspect 13: The apparatus of any of Aspects 8 to 12, wherein, to obtain the behavioral information associated with the user of the apparatus, the at least one processor is configured to detect, in one or more images obtained by the apparatus, one or more behavioral artifacts associated with the behavior.

Aspect 14: The apparatus of any of Aspects 8 to 13, wherein, to generate the intervention, the at least one processor is configured to display virtual content on a display of the apparatus, wherein a real-world environment is viewable through the display of the apparatus as the virtual content is displayed by the display.

Aspect 15: The apparatus of any of Aspects 8 to 14, wherein the apparatus is an extended reality (XR) device.

Aspect 16: A non-transitory computer-readable storage medium having stored thereon instructions which, when executed by one or more processors, cause the one or more processors to perform any of the operations of aspects 1 to 14.

Aspect 17: An apparatus comprising means for performing any of the operations of aspects 1 to 15.

Aspect 18: A method of generating one or more interventions, the method comprising: obtaining, by a server, first intervention information associated with a first user and a first intervention; updating, based on the first intervention information, one or more parameters of an intervention library, wherein the one or more parameters of the intervention library are based at least in part on second intervention information associated with a second user and a second intervention; and determining a third intervention for a third user based on the updated one or more parameters of the intervention library.

Aspect 19: The method of Aspect 18, wherein the first intervention information associated with the first user comprises at least one of an intervention type, an indication of an effectiveness of the first intervention, an intervention context associated with the first intervention, and one or more characteristics associated with the first user.

Aspect 20: The method of any of Aspects 18 to 19, wherein determining the third intervention for the third user based on the updated one or more parameters of the intervention library comprises: obtaining, by the server, third intervention information associated with the third user; determining a correlation between the third intervention information associated with the third user and fourth intervention information associated with the intervention library; determining, based on the correlation between the third intervention information and the fourth intervention information exceeding a correlation threshold, the third intervention; and sending, to a device associated with the third user, the third intervention.

Aspect 21: The method of any of Aspects 18 to 20, wherein the first intervention information comprises contextual information associated with the first intervention.

Aspect 22: The method of any of Aspects 18 to 21, wherein the contextual information associated with the first intervention comprises at least one of a time of day, one or more actions by the first user prior to the first intervention, the first intervention information associated with the first user, a location the first user, and a proximity of the first user to one or more individuals.

Aspect 23: The method of any of Aspects 18 to 22, wherein the first intervention information comprises one or more characteristics associated with the first user.

Aspect 24: The method of any of Aspects 18 to 23, wherein the one or more characteristics associated with the first user comprise at least one of gender, age, family status, target behavior, country, culture, locale, and personality type.

Aspect 25: The method of any of Aspects 18 to 24, further comprising: obtaining, by the server, fifth behavioral information associated with a fifth user and a fifth behavior; updating, based on the fifth behavioral information, one or more parameters of a behavior library, wherein the one or more parameters of the behavior library are based at least in part on sixth behavioral information associated with a sixth user; and determining one or more behavior parameters for a seventh user based on the updated one or more parameters of the intervention library.

Aspect 26: The method of any of Aspects 18 to 25, wherein the one or more behavior parameters for the seventh user comprise at least one of behavioral triggers, pre-behaviors, and behavioral artifacts associated with the fifth behavior.

Aspect 27: The method of any of Aspects 18 to 26, wherein the one or more behavior parameters for the seventh user comprise one or more weightings associated with determining a likelihood that the seventh user will perform or not perform the fifth behavior.

Aspect 28: The method of any of Aspects 18 to 27, wherein the fifth behavioral information comprises one or more characteristics associated with the fifth user.

Aspect 29: The method of any of Aspects 18 to 28, wherein the fifth behavioral information comprises contextual information associated with the fifth behavior.

Aspect 30: A system for generating one or more interventions, comprising: at least one memory; and at least one processor coupled to the at least one memory, the at least one processor configured to: obtain first intervention information associated with a first user and a first intervention; update, based on the first intervention information, one or more parameters of an intervention library, wherein the one or more parameters of the intervention library are based at least in part on second intervention information associated with a second user and a second intervention; and determine a third intervention for a third user based on the updated one or more parameters of the intervention library.

Aspect 31: The system of Aspect 30, wherein the first intervention information associated with the first user comprises at least one of an intervention type, an indication of an effectiveness of the first intervention, an intervention context associated with the first intervention, and one or more characteristics associated with the first user.

Aspect 32: The system of any of Aspects 30 to 31, wherein, to determine the third intervention for the third user based on the updated one or more parameters of the intervention library, the at least one processor is configured to: obtain third intervention information associated with the third user; determine a correlation between the third intervention information associated with the third user and fourth intervention information associated with the intervention library; determine, based on the correlation between the third intervention information and the fourth intervention information exceeding a correlation threshold, the third intervention; and send, to a device associated with the third user, the third intervention.

Aspect 33: The system of any of Aspects 30 to 32, wherein the first intervention information comprises contextual information associated with the first intervention.

Aspect 34: The system of any of Aspects 30 to 33, wherein the contextual information associated with the first intervention comprises at least one of a time of day, one or more actions by the first user prior to the first intervention, the first intervention information associated with the first user, a location the first user, and a proximity of the first user to one or more individuals.

Aspect 35: The system of any of Aspects 30 to 34, wherein the first intervention information comprises one or more characteristics associated with the first user.

Aspect 36: The system of any of Aspects 30 to 35, wherein the one or more characteristics associated with the first user comprise at least one of gender, age, family status, target behavior, country, culture, locale, and personality type.

Aspect 37: The system of any of Aspects 30 to 36, wherein the at least one processor is configured to: obtain fifth behavioral information associated with a fifth user and a fifth behavior; update, based on the fifth behavioral information, one or more parameters of a behavior library, wherein the one or more parameters of the behavior library are based at least in part on sixth behavioral information associated with a sixth user; and determine one or more behavior parameters for a seventh user based on the updated one or more parameters of the intervention library.

Aspect 38: The system of any of Aspects 30 to 37, wherein the one or more behavior parameters for the seventh user comprise at least one of behavioral triggers, pre-behaviors, and behavioral artifacts associated with the fifth behavior.

Aspect 39: The system of any of Aspects 30 to 38, wherein the one or more behavior parameters for the seventh user comprise one or more weightings associated with determining a likelihood that the sixth user will perform or not perform the fifth behavior.

Aspect 40: The system of any of Aspects 30 to 39, wherein the fifth behavioral information comprises one or more characteristics associated with the fifth user.

Aspect 41: The system of any of Aspects 30 to 40, wherein the fifth behavioral information comprises contextual information associated with the fifth behavior.

Aspect 42: The system of any of Aspects 30 to 41, wherein the system includes at least one server.

Aspect 43: A non-transitory computer-readable storage medium having stored thereon instructions which, when executed by one or more processors, cause the one or more processors to perform any of the operations of aspects 18 to 42.

Aspect 44: An apparatus comprising means for performing any of the operations of aspects 18 to 42.

Aspect 45: A method comprising operations according to any of Aspects 1-7 and any of Aspects 18-29.

Aspect 46: An apparatus for performing temporal blending for one or more frames. The apparatus includes a memory (e.g., implemented in circuitry) configured to store one or more frames and one or more processors (e.g., one processor or multiple processors) coupled to the memory. The one or more processors are configured to perform operations according to any of Aspects 1-7 and any of Aspects 18-29.

Aspect 47: A computer-readable storage medium storing instructions that, when executed by one or more processors, cause the one or more processors to perform operations according to any of Aspects 1-7 and any of Aspects 18-29.

Aspect 48: An apparatus comprising means for performing operations according to any of Aspects 1-7 and any of Aspects 18-29.

1. A method of generating one or more interventions, the method comprising:
obtaining, by an extended reality (XR) device, behavioral information associated with a user of the XR device;
determining, by the XR device based on the behavioral information, a likelihood of the user engaging in a behavior;
determining, by the XR device based on the determined likelihood exceeding a likelihood threshold, an intervention;
generating, by the XR device, the intervention;
determining, subsequent to generating the intervention, whether the user engaged in the behavior;
determining an effectiveness of the intervention based on whether the user engaged in the behavior; and
sending, to a server, an indication of the effectiveness of the intervention for use in determining interventions for one or more additional users.

2. The method of claim 1, further comprising:
sending, to the server, contextual information associated with the intervention, wherein the contextual information associated with the intervention comprises at least one of a time of day, one or more actions by the user of the XR device prior to the intervention, the behavioral information associated with the user of the XR device, a location of the user of the XR device, and a proximity of the user of the XR device to one or more individuals.

3. The method of claim 1, further comprising:
sending, to the server, one or more characteristics associated with the user of the XR device, wherein the one or more characteristics associated with the user of the XR device comprise at least one of gender, age, family status, target behavior, country, culture, locale, personality type, one or more health conditions, one or more dietary restrictions, and one or more physical capabilities.

4. The method of claim 1, wherein obtaining the behavioral information associated with the user of the XR device includes at least one of:
determining one or more behavioral triggers that are predictive of the behavior, wherein the one or more behavioral triggers include at least one of a stress level of the user, a heart rate of the user, an object within a field of view of the XR device, a location at which the user is located, a time at which the behavioral information is obtained, one or more people in proximity to the user, and an activity in which the user is engaged.;
determining one or more pre-behaviors indicative of a likelihood of the user engaging in the behavior; or
detecting, in one or more images obtained by the XR device, one or more behavioral artifacts associated with the behavior.

5. (canceled)

6. (canceled)

7. The method of claim 1, wherein generating the intervention comprises displaying virtual content on a display of the XR device, wherein a real-world environment is viewable through the display of the XR device as the virtual content is displayed by the display.

8. An apparatus for generating one or more interventions, comprising:
at least one memory; and
at least one processor coupled to the at least one memory, the at least one processor configured to:
obtain behavioral information associated with a user of the apparatus;
determine, based on the behavioral information, a likelihood of the user engaging in a behavior;
determine, based on the determined likelihood exceeding a likelihood threshold, an intervention associated with the behavior;
generate the intervention;
determine, subsequent to outputting the intervention, whether the user engaged in the behavior;
determine an effectiveness of the intervention based on whether the user engaged in the behavior; and
send, to a server, an indication of the effectiveness of the intervention for use in determining interventions for one or more additional users.

9. The apparatus of claim 8, wherein the at least one processor is configured to:
send, to the server, contextual information associated with the intervention, wherein the contextual information associated with the intervention comprises at least one of a time of day, one or more actions by the user of the apparatus prior to the intervention, the behavioral information associated with the user of the apparatus, a location of the user of the apparatus, and a proximity of the user of the apparatus to one or more individuals.

10. The apparatus of claim 8, wherein the at least one processor is configured to:
send, to the server, one or more characteristics associated with the user of the apparatus, wherein the one or more characteristics associated with the user of the apparatus comprise at least one of gender, age, family status, target behavior, country, culture, locale, personality type, one or more health conditions, one or more dietary restrictions, and one or more physical capabilities.

11. The apparatus of claim 8, wherein, to obtain the behavioral information associated with the user of the apparatus, the at least one processor is configured to determine one or more behavioral triggers that are predictive of the behavior, wherein the one or more behavioral triggers include at least one of a stress level of the user, a heart rate of the user, an object within a field of view of the apparatus, a location at which the user is located, a time at which the behavioral information is obtained, one or more people in proximity to the user, and an activity in which the user is engaged.

12. The apparatus of claim 11, wherein, to obtain the behavioral information associated with the user of the apparatus, the at least one processor is configured to determine one or more pre-behaviors indicative of a likelihood of the user engaging in the behavior.

13. The apparatus of claim 8, wherein, to obtain the behavioral information associated with the user of the apparatus, the at least one processor is configured to detect, in one or more images obtained by the apparatus, one or more behavioral artifacts associated with the behavior.

14. The apparatus of claim 8, wherein, to generate the intervention, the at least one processor is configured to display virtual content on a display of the apparatus, wherein a real-world environment is viewable through the display of the apparatus as the virtual content is displayed by the display.

15. The apparatus of claim 8, wherein the apparatus is an extended reality (XR) device.

16. A method of generating one or more interventions, the method comprising:
obtaining, by a server, first intervention information associated with a first user and a first intervention;
updating, based on the first intervention information, one or more parameters of an intervention library, wherein the one or more parameters of the intervention library are based at least in part on second intervention information associated with a second user and a second intervention; and
determining a third intervention for a third user based on the updated one or more parameters of the intervention library.

17. The method of claim 16, wherein the first intervention information associated with the first user comprises at least one of an intervention type, an indication of an effectiveness of the first intervention, an intervention context associated with the first intervention, and one or more characteristics associated with the first user.

18. The method of claim 16, wherein determining the third intervention for the third user based on the updated one or more parameters of the intervention library comprises:
obtaining, by the server, third intervention information associated with the third user;
determining a correlation between the third intervention information associated with the third user and fourth intervention information associated with the intervention library;
determining, based on the correlation between the third intervention information and the fourth intervention information exceeding a correlation threshold, the third intervention; and
sending, to a device associated with the third user, the third intervention.

19. The method of claim 16, wherein the first intervention information comprises at least one of contextual information associated with the first intervention or one or more characteristics associated with the first user.

20. (canceled)

21. (canceled)

22. (canceled)

23. (Canceled)

24. (canceled)

25. (canceled)

26. (canceled)

27. (canceled)

28. A system for generating one or more interventions, comprising:
at least one memory; and
at least one processor coupled to the at least one memory, the at least one processor configured to:
obtain first intervention information associated with a first user and a first intervention;
update, based on the first intervention information, one or more parameters of an intervention library, wherein the one or more parameters of the intervention library are based at least in part on second intervention information associated with a second user and a second intervention; and
determine a third intervention for a third user based on the updated one or more parameters of the intervention library.

29. The system of claim 28, wherein the first intervention information associated with the first user comprises at least one of an intervention type, an indication of an effectiveness of the first intervention, an intervention context associated with the first intervention, and one or more characteristics associated with the first user.

30. The system of claim 28, wherein, to determine the third intervention for the third user based on the updated one or more parameters of the intervention library, the at least one processor is configured to:
obtain third intervention information associated with the third user;
determine a correlation between the third intervention information associated with the third user and fourth intervention information associated with the intervention library;
determine, based on the correlation between the third intervention information and the fourth intervention information exceeding a correlation threshold, the third intervention; and
send, to a device associated with the third user, the third intervention.

31. The system of claim 28, wherein the first intervention information comprises contextual information associated with the first intervention.

32. The system of claim 31, wherein the contextual information associated with the first intervention comprises at least one of a time of day, one or more actions by the first user prior to the first intervention, the first intervention information associated with the first user, a location the first user, and a proximity of the first user to one or more individuals.

33. The system of claim 28, wherein the first intervention information comprises one or more characteristics associated with the first user.

34. The system of claim 33, wherein the one or more characteristics associated with the first user comprise at least one of gender, age, family status, target behavior, country, culture, locale, and personality type.

35. The system of claim 28, wherein the at least one processor is configured to:
obtain fifth behavioral information associated with a fifth user and a fifth behavior;
update, based on the fifth behavioral information, one or more parameters of a behavior library, wherein the one or more parameters of the behavior library are based at least in part on sixth behavioral information associated with a sixth user; and
determine one or more behavior parameters for a seventh user based on the updated one or more parameters of the intervention library.

36. The system of claim 35, wherein the one or more behavior parameters for the seventh user comprise at least one of behavioral triggers, pre-behaviors, and behavioral artifacts associated with the fifth behavior.

37. The system of claim 35, wherein the one or more behavior parameters for the seventh user comprise one or more weightings associated with determining a likelihood that the sixth user will perform or not perform the fifth behavior.

38. The system of claim 35, wherein the fifth behavioral information comprises one or more characteristics associated with the fifth user.

39. The system of claim 35, wherein the fifth behavioral information comprises contextual information associated with the fifth behavior. server.

40. The system of claim 28, wherein the system includes at least one
